SEQUENCE LISTING (58 sequences)

SEQ ID NO: 1 - protein, 16 aa, Artificial Sequence
  repeating sequence
LEGSNTIFVRGDETKT

SEQ ID NO: 2 - protein, 86 aa, Bacteriophage T4
VEGNGTILVKGNVTIIVEGNADITVKGDATTLVEGNQTNTVNGNLSWKVAGTVDWDVGGDWTEKMASMSSISSGQYTIDGSRIDIG

SEQ ID NO: 3 - protein, 125 aa, Bacteriophage P2
VEGNGTILVKGNVTIIVEGNADITVKGDATTLDALHIRFPDGAVIEYEPETSALTVSGIKTASVTASGSVTATVPVVMVKASTRVTLDTPEVVCTNRLITGTLEVQKGGTMRGNIEHTGGELSSN

SEQ ID NO: 4 - protein, 107 aa, Bacteriophage Mu
VEGNGTILVKGNVTIIVEGNADITVKGDATTLGDAGIYHHEGHRIRLTKDGRCIITCKTVEVYADESMTVDTPRTTFTGDVEIQKGLGVKGKSQFDSNITAPDAIIN

SEQ ID NO: 5 - protein, 110 aa, Bacteriophage phi 92
VEGNGTILVKGNVTIIVEGNADITVKGDATTLEKVIISNNKQTYASFDPNGNISVYNTQGMKIDMTPNSIVLTDAGGGKLTLQGGTMTYKGGTVNLNGLTITPDGRMTDS

SEQ ID NO: 6 - protein, 27 aa, Bacteriophage T4
GYIPEAPRDGQAYVRKDGEWVLLSTFL

SEQ ID NO: 7 - protein, 21 aa, Bacteriophage P2
GKVLHTHKHPGDSGGTTGSPL

SEQ ID NO: 8 - protein, 23 aa, Bacteriophage Mu
GKSTDKHIHRGDSGGTTGPMQLE

SEQ ID NO: 9 - protein, 30 aa, Bacteriophage phi 92
GGIGLHTHTHPVRGVETGGSTVTSDKPNGG

SEQ ID NO: 10 - protein, 133 aa, Human norovirus LM14-2 strain
MGKKGKNKSGRGKKHTAFSSKGLSDEEYDEYKRIREERNGKYSIEEYLQDRDRYYEEVAIARATEEDFCEEEEAKIRQRIFRPTRKQRKEERASLGLVTGSEIRKRNPEDFKPKGKLWADDDRSVDYNEKLNF

SEQ ID NO: 11 - DNA, 12774 bp, Artificial Sequence
  plasmid pHuNoV-LM14-2F
tcgaggccgcggccgcggtcgactctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgacgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcaagcttgcctcgaatcacaagtttgtacaaaaaagcaggcttcgaaggagatagaaccgtgaatgaagatggcgtctaacgacgcttccgctgccgctgttgccaacagcaacaacgacatcgcaaaatcttcaagtgacggtgtgctttctaacatggctgtcacttttaagcgggccctcggggcgcggcctaaacagccgcccccgaaggaaataccacctagacccccgcgaccacccacaccagaattggtcaaaaagatccctcctcccccacccaacggggaggatgaactagtggtctcttacagcgccaaagatggcgtttccggactgcctgagctcaccactgtcagacaaccggaagaaaccaacacggcgttcagtgtccccccactcaaccaaagggagagcagggacgccaaggagccactaactggaacaatcattgaaatgtgggatggagaaatctaccattacggcctgtacgtggaacgaggtcttatacttggtgtgcacaagccaccggcagccattagccttgccaaggtcgagctagcaccgctctctttgttctggagacctgtatacaccccccagtatctcatctctccagacactcttaggagattacatggagagtcattcccctacactgcatttgacaacaattgctacgccttttgttgttgggtattagacctaaacgactcatggctaagcaggagaatgattcagagaacaacaggcttcttcaggccgtaccaggattggaacaggaaacccctccccactatggatgattccaaattaaagaaggtagccaacatattcttgtgcactttgtcttcactattcaccagacccattaaggacataatagggaagttgaaacctcttaacatccttaacattctggctacatgtgattggaccttcgcaggcatagtggaatcctcaatactcttggcagaactctttggagttttctggacacccccagatgtgtctgcgatgatcgcccccttgctaggtgattatgaactgcaaggacctgaggaccttgcagtggaactggtcccaatagtaatgggggggataggtttggtgctaggatttaccaaagagaaaatcggaaagatgctatcatccgctgcatccactttaagagcttgtaaagaccttggtgcatacggactggaaatcttaaaattggtcatgaagtggttcttcccaaagaaagaggaagcaaatgaactggctatggtgagatccatcgaggatgcagtactagacctcgaggcaattgaaaacaaccacatgaccaccctactcaaagacaaagacagcttggcaacctacatgagaacccttgaccttgaggaggagaaagccagaaaactctcaaccaaatctgcttcacccgatattgtgggcacaatcaactctcttctggcaagaatcgctgctgcacgctccctagtgcatcgggcgaaagaagagctctccagcaggccgagacctgtcgttgtgatgatatcgggaagaccagggatagggaaaactcaccttgccagggagctggccaagaagatcgcggcctccctcacaggggaccagcgtgtgggtcttatcccgcgcaatggtgtcgaccactgggacgcatacaagggcgaaagagttgtcctatgggacgactatggaatgagcaaccccatccatgatgccctcaggttgcaggagcttgctgacacttgccccctcacgctaaattgtgacagaattgagaacaaagggaaagtctttgacagtgatgccataattatcaccaccaatctggccaacccagcaccactggattatgtcaactttgaagcgtgctcgagacgcattgatttcctcgtgtacgcagaagcccctgaggtggagaaggcaaagcgcgacttcccaggtcaacctgacatgtggaagaacgctttcagtcctgacttctcacacataaaactgtcattggctccacagggtggttttgacaagaacggcaacaccccgcatggaaaaggggtcatgaagaccctcaccactggctccctcatcgcccgagcatcagggttactccatgagaggctagatgaatatgaactgcaaggcccagccctcaccactttcaactttgaccgcaacaagatacttgcttttagacagcttgctgctgaaaacaagtatgggctgatggacacaatgagagttggaaaacagctcaaggatgtcaagaccatgtcagacctcaaacaagcacttaagaacatcgcgatcaagaagtgccagatagtgtacaatggtggcacctacacacttgaggctgatggcaagggtagtgtgaaagttgacaaagtgcaaagtgccactgtgcagaccaacaatgaactagccggtgccctacaccacctaaggtgcgctagaatcagatactatgttaagtgcgtccaggaggcactgtattccatcatccaaatcgctggggctgcattcgtcaccacgcgcatcgccaagcgcatgaacatacagaatctctggtccaagccacaggtggaagacacagaagagatggccaacaaagatggttgcctaaaacccaaagatgatgaagagtttgtcgtctcatccgacgacatcaaaactgagggcaagaaagggaagaacaagtccggccgtggcaagaagcacacagccttttcaagtaaagggctcagtgatgaggagtacgatgagtacaagagaatcagagaagaaaggaatggtaagtactccatagaagagtaccttcaggacagagacaggtactacgaggaggtggccattgccagggcaaccgaagaggacttctgtgaagaagaagaggccaaaatccggcagagaatttttagaccaacaaggaaacaacgcaaagaagagagggcctctctcggcttggtcacaggctctgaaatcaggaagagaaacccagaagacttcaaacccaagggaaagctgtgggctgatgacgacagaagtgttgactacaatgagaaactcaactttgaggccccaccaagcatctggtcgcggatagtcaactttggttcaggctggggcttctgggtctcccccagtctgtttataacatcaacccatgtcataccccaaggtgcaaaagagttcttcggagtccctatcaagcaaatccagatacacaagtcaggtgaattctgccggttgaggttcccaaagccaatcagaactgatgtgacgggcatgattctagaagaaggtgcgcccgaggggaccgtggccacactgctcatcaagagaccaactggagagctcatgcctctggcagccagaatggggacccatgcaaccatgaaaattcaggggcgcacagttggagggcaaatgggtatgctcctgacaggatccaacgccaagagtatggacctaggcacaacaccaggcgactgcggctgcccctacatctacaagagggggaatgactatgtggtcataggagtccatacggccgctgcccgtggaggaaacactgtcatatgtgccacccaggggagtgagggagaagccacacttgaaggaggtgacagtaaagggacatactgtggcgcaccaatcttgggcccagggagcgctccgaatctcagtaccaagactaagttttggaggtcatccacaacaccactcccacctggcacctacgaaccagcctacctcggtggcaaagaccctagagtcaagggtggcccttcattgcaacaagttatgagggaccagctgaagccattcacagaacccagaggcaaaccaccaagaccaaatgtgttggaagctgccaagaaaaccatcatcaatgtccttgagcaaacgattgatccaccccaaaaatggtcatttgcgcaagcttgcgcatcccttgacaaaaccacctccagcggccacccgcaccacatgcggaaaaacgactgttggaatggggagtccttcacaggaaaattggctgatcaagcctccaaggccaacctaatgtttgaagagggaaagaacatgactccagtctacacaggtgcacttaaagatgagttggtaaagaccgataaagtttatggtaaggtcaagaagaggcttctgtggggttcagatctggcgaccatgatacggtgcgcccgagcttttggaggccttatggatgaactcaaggcacactgtgtcacacttcctgtcagagttggtatgaacatgaatgaggatggccccatcatctttgagaagcactccagatatagatatcactatgatgctgattattcccggtgggactcaacacaacaaagggatgtgctagcagcagcactagaaatcatggttaagttctctccagaaccacacctggcccagatagttgcagaagacctcctttcccctagcgtgatggatgtaggtgactttcaaatatcaataagtgagggtctcccctctggggtaccttgtacctcccagtggaattccatcgcccactggctcctcactctgtgtgcactctctgaagtcacggacctgtcccctgatatcattcaggccaactcccttttctccttctatggtgatgatgagattgtaagcacagacataaagttggacccagagaagctgacagcaaaactcaaggagtacgggctgaaaccaacccgccccgacaaaactgaaggaccccttgttatctctgaagacctggatggcctgacattcctccggagaactgtgacccgtgatccagctggctggtttggaaaattggaacaaagttcaattctcaggcaaatgtactggaccaggggtcccaaccatgaagatccatttgaaacaatgataccacactcccaaagacccatacaattgatgtccctgctgggcgaggctgcactccacggcccggcattctatagcaaaattagcaaattagtcattgcagagttgaaggaaggtggcatggatttttacgtacccagacaagagccaatgttcagatggatgagattctcagatctgagcacgtgggagggcgatcgcaatctggctcccagttttgtgaatgaagatggcgtcgagtgacgccaacccatctgatgggtccgcagccaacctcgtcccagaggtcaacaatgaggttatggctctggagcccgttgttggtgccgccattgcggcacctgtagcgggccaacaaaatgtaattgacccctggattagaaataattttgtacaagcccctggtggagagtttacagtatcccctagaaacgctccaggtgaaatactatggagcgcgcccttgggccctgatctaaatccctacctatcccatttggccagaatgtacaatggttatgcaggtggttttgaagtgcaggtaattctcgcggggaacgcgttcaccgccgggaaggtcatatttgcagcagtcccaccaaattttccaactgaaggcttgagccccagccaggtcactatgttcccccatatagtagtagatgttaggcaactagaacctgtgttgattcccttacccgatgttaggaataatttctatcattacaatcaatcaaatgaccccaccattaagttgatagcaatgttgtatacaccacttagggctaataatgctggggatgatgtcttcacagtctcttgccgagttctcacgagaccatcccccgattttgatttcatatttctagtgccacccacagttgagtcaagaactaaaccattctctgtcccagttttaactgttgaggagatgaccaattcaagattccccattcctttggaaaagttgttcacgggtcccagcagtgcctttgttgtccaaccacaaaacggtaggtgcacgactgatggcgtgctcctaggcaccacccaactgtctcctgtcaacatctgcaccttcagaggagatgtcacccatatcacaggtagtcgtaactacacaatgaatttggcttctcaaaattggagcaattatgacccaacagaagaaatcccagcccctctaggaactccagattttgtggggaagattcaaggcgtgctcacccaaaccacaaggacagatggctcaacacgcggccacaaagccacagtgtacactgggagcgccggctttgctccaaaactgggtagagttcaatttgaaactgacacagaccatgattttgaagctaaccaaaacacaaagttcaccccagttggtgtcatccaagatggtagcaccacccaccgaaatgaaccccaacagtgggtgctcccaagttactcaggcagaaatactcctaatgtgcatctagcccccgctgtagcccccacttttccgggtgagcaacttctcttctttagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcactataatggctggagctttctttgctggattggcatctgatgtccttggctctggacttggttcccttatcaatgctggggctggggccatcaaccaaaaagttgagtttgaaaataacagaaaattgcaacaagcatccttccaatttagcagcaatctacaacaggcttcctttcaacatgacaaagagatgctccaagcacaaattgaggccaccaaaaggctacaacaggaaatgatgaaagttaagcaggcaatgctcctagagggtgggttctctgagacagatgcagcccgcggggcaatcaacgcccccatgacaaaaactttggactggagcgggacaaggtactgggctcccgatgctaggactacaacatacaatgcaggccgcttttccacccctcaaccatcgggggcactgccaggaagagctaatcttagggatgctgtccctgctcggggttcctccagtaagtcttctaattcttctactgccacttctgtgtactcaaatcaaactacttcaacgagacttggttctacagctggttctggtaccagtgtctcgagcttcccgtcaactgcaaggactaggagctgggttgaggatcaaagtaggaatttgtcacctttcatgaggggggcccacaacatatcgtttgtcaccccaccatctagcagatcctctagccaaggcacagtctcaaccgtgcctaaagagattttggactcctggactggcgctttcaacacgcgcaggcagccactcttcgctcacattcgtaagcggggggagtcacgggcgtaatgtgaaaagacaaaattgattatctttcttttctttagtgtcttttaaaaaaaaaaaaaaaaaaaaaaaaaaaaaaaaaaaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagggcggggatccggctgctaacaaagcccgaaaggacccagctttcttgtacaaagtggtgat

SEQ ID NO: 12 - DNA, 7639 bp, Artificial Sequence
  cDNA part in plasmid pHuNoV-LM14-2F
gtgaatgaagatggcgtctaacgacgcttccgctgccgctgttgccaacagcaacaacgacatcgcaaaatcttcaagtgacggtgtgctttctaacatggctgtcacttttaagcgggccctcggggcgcggcctaaacagccgcccccgaaggaaataccacctagacccccgcgaccacccacaccagaattggtcaaaaagatccctcctcccccacccaacggggaggatgaactagtggtctcttacagcgccaaagatggcgtttccggactgcctgagctcaccactgtcagacaaccggaagaaaccaacacggcgttcagtgtccccccactcaaccaaagggagagcagggacgccaaggagccactaactggaacaatcattgaaatgtgggatggagaaatctaccattacggcctgtacgtggaacgaggtcttatacttggtgtgcacaagccaccggcagccattagccttgccaaggtcgagctagcaccgctctctttgttctggagacctgtatacaccccccagtatctcatctctccagacactcttaggagattacatggagagtcattcccctacactgcatttgacaacaattgctacgccttttgttgttgggtattagacctaaacgactcatggctaagcaggagaatgattcagagaacaacaggcttcttcaggccgtaccaggattggaacaggaaacccctccccactatggatgattccaaattaaagaaggtagccaacatattcttgtgcactttgtcttcactattcaccagacccattaaggacataatagggaagttgaaacctcttaacatccttaacattctggctacatgtgattggaccttcgcaggcatagtggaatcctcaatactcttggcagaactctttggagttttctggacacccccagatgtgtctgcgatgatcgcccccttgctaggtgattatgaactgcaaggacctgaggaccttgcagtggaactggtcccaatagtaatgggggggataggtttggtgctaggatttaccaaagagaaaatcggaaagatgctatcatccgctgcatccactttaagagcttgtaaagaccttggtgcatacggactggaaatcttaaaattggtcatgaagtggttcttcccaaagaaagaggaagcaaatgaactggctatggtgagatccatcgaggatgcagtactagacctcgaggcaattgaaaacaaccacatgaccaccctactcaaagacaaagacagcttggcaacctacatgagaacccttgaccttgaggaggagaaagccagaaaactctcaaccaaatctgcttcacccgatattgtgggcacaatcaactctcttctggcaagaatcgctgctgcacgctccctagtgcatcgggcgaaagaagagctctccagcaggccgagacctgtcgttgtgatgatatcgggaagaccagggatagggaaaactcaccttgccagggagctggccaagaagatcgcggcctccctcacaggggaccagcgtgtgggtcttatcccgcgcaatggtgtcgaccactgggacgcatacaagggcgaaagagttgtcctatgggacgactatggaatgagcaaccccatccatgatgccctcaggttgcaggagcttgctgacacttgccccctcacgctaaattgtgacagaattgagaacaaagggaaagtctttgacagtgatgccataattatcaccaccaatctggccaacccagcaccactggattatgtcaactttgaagcgtgctcgagacgcattgatttcctcgtgtacgcagaagcccctgaggtggagaaggcaaagcgcgacttcccaggtcaacctgacatgtggaagaacgctttcagtcctgacttctcacacataaaactgtcattggctccacagggtggttttgacaagaacggcaacaccccgcatggaaaaggggtcatgaagaccctcaccactggctccctcatcgcccgagcatcagggttactccatgagaggctagatgaatatgaactgcaaggcccagccctcaccactttcaactttgaccgcaacaagatacttgcttttagacagcttgctgctgaaaacaagtatgggctgatggacacaatgagagttggaaaacagctcaaggatgtcaagaccatgtcagacctcaaacaagcacttaagaacatcgcgatcaagaagtgccagatagtgtacaatggtggcacctacacacttgaggctgatggcaagggtagtgtgaaagttgacaaagtgcaaagtgccactgtgcagaccaacaatgaactagccggtgccctacaccacctaaggtgcgctagaatcagatactatgttaagtgcgtccaggaggcactgtattccatcatccaaatcgctggggctgcattcgtcaccacgcgcatcgccaagcgcatgaacatacagaatctctggtccaagccacaggtggaagacacagaagagatggccaacaaagatggttgcctaaaacccaaagatgatgaagagtttgtcgtctcatccgacgacatcaaaactgagggcaagaaagggaagaacaagtccggccgtggcaagaagcacacagccttttcaagtaaagggctcagtgatgaggagtacgatgagtacaagagaatcagagaagaaaggaatggtaagtactccatagaagagtaccttcaggacagagacaggtactacgaggaggtggccattgccagggcaaccgaagaggacttctgtgaagaagaagaggccaaaatccggcagagaatttttagaccaacaaggaaacaacgcaaagaagagagggcctctctcggcttggtcacaggctctgaaatcaggaagagaaacccagaagacttcaaacccaagggaaagctgtgggctgatgacgacagaagtgttgactacaatgagaaactcaactttgaggccccaccaagcatctggtcgcggatagtcaactttggttcaggctggggcttctgggtctcccccagtctgtttataacatcaacccatgtcataccccaaggtgcaaaagagttcttcggagtccctatcaagcaaatccagatacacaagtcaggtgaattctgccggttgaggttcccaaagccaatcagaactgatgtgacgggcatgattctagaagaaggtgcgcccgaggggaccgtggccacactgctcatcaagagaccaactggagagctcatgcctctggcagccagaatggggacccatgcaaccatgaaaattcaggggcgcacagttggagggcaaatgggtatgctcctgacaggatccaacgccaagagtatggacctaggcacaacaccaggcgactgcggctgcccctacatctacaagagggggaatgactatgtggtcataggagtccatacggccgctgcccgtggaggaaacactgtcatatgtgccacccaggggagtgagggagaagccacacttgaaggaggtgacagtaaagggacatactgtggcgcaccaatcttgggcccagggagcgctccgaatctcagtaccaagactaagttttggaggtcatccacaacaccactcccacctggcacctacgaaccagcctacctcggtggcaaagaccctagagtcaagggtggcccttcattgcaacaagttatgagggaccagctgaagccattcacagaacccagaggcaaaccaccaagaccaaatgtgttggaagctgccaagaaaaccatcatcaatgtccttgagcaaacgattgatccaccccaaaaatggtcatttgcgcaagcttgcgcatcccttgacaaaaccacctccagcggccacccgcaccacatgcggaaaaacgactgttggaatggggagtccttcacaggaaaattggctgatcaagcctccaaggccaacctaatgtttgaagagggaaagaacatgactccagtctacacaggtgcacttaaagatgagttggtaaagaccgataaagtttatggtaaggtcaagaagaggcttctgtggggttcagatctggcgaccatgatacggtgcgcccgagcttttggaggccttatggatgaactcaaggcacactgtgtcacacttcctgtcagagttggtatgaacatgaatgaggatggccccatcatctttgagaagcactccagatatagatatcactatgatgctgattattcccggtgggactcaacacaacaaagggatgtgctagcagcagcactagaaatcatggttaagttctctccagaaccacacctggcccagatagttgcagaagacctcctttcccctagcgtgatggatgtaggtgactttcaaatatcaataagtgagggtctcccctctggggtaccttgtacctcccagtggaattccatcgcccactggctcctcactctgtgtgcactctctgaagtcacggacctgtcccctgatatcattcaggccaactcccttttctccttctatggtgatgatgagattgtaagcacagacataaagttggacccagagaagctgacagcaaaactcaaggagtacgggctgaaaccaacccgccccgacaaaactgaaggaccccttgttatctctgaagacctggatggcctgacattcctccggagaactgtgacccgtgatccagctggctggtttggaaaattggaacaaagttcaattctcaggcaaatgtactggaccaggggtcccaaccatgaagatccatttgaaacaatgataccacactcccaaagacccatacaattgatgtccctgctgggcgaggctgcactccacggcccggcattctatagcaaaattagcaaattagtcattgcagagttgaaggaaggtggcatggatttttacgtacccagacaagagccaatgttcagatggatgagattctcagatctgagcacgtgggagggcgatcgcaatctggctcccagttttgtgaatgaagatggcgtcgagtgacgccaacccatctgatgggtccgcagccaacctcgtcccagaggtcaacaatgaggttatggctctggagcccgttgttggtgccgccattgcggcacctgtagcgggccaacaaaatgtaattgacccctggattagaaataattttgtacaagcccctggtggagagtttacagtatcccctagaaacgctccaggtgaaatactatggagcgcgcccttgggccctgatctaaatccctacctatcccatttggccagaatgtacaatggttatgcaggtggttttgaagtgcaggtaattctcgcggggaacgcgttcaccgccgggaaggtcatatttgcagcagtcccaccaaattttccaactgaaggcttgagccccagccaggtcactatgttcccccatatagtagtagatgttaggcaactagaacctgtgttgattcccttacccgatgttaggaataatttctatcattacaatcaatcaaatgaccccaccattaagttgatagcaatgttgtatacaccacttagggctaataatgctggggatgatgtcttcacagtctcttgccgagttctcacgagaccatcccccgattttgatttcatatttctagtgccacccacagttgagtcaagaactaaaccattctctgtcccagttttaactgttgaggagatgaccaattcaagattccccattcctttggaaaagttgttcacgggtcccagcagtgcctttgttgtccaaccacaaaacggtaggtgcacgactgatggcgtgctcctaggcaccacccaactgtctcctgtcaacatctgcaccttcagaggagatgtcacccatatcacaggtagtcgtaactacacaatgaatttggcttctcaaaattggagcaattatgacccaacagaagaaatcccagcccctctaggaactccagattttgtggggaagattcaaggcgtgctcacccaaaccacaaggacagatggctcaacacgcggccacaaagccacagtgtacactgggagcgccggctttgctccaaaactgggtagagttcaatttgaaactgacacagaccatgattttgaagctaaccaaaacacaaagttcaccccagttggtgtcatccaagatggtagcaccacccaccgaaatgaaccccaacagtgggtgctcccaagttactcaggcagaaatactcctaatgtgcatctagcccccgctgtagcccccacttttccgggtgagcaacttctcttctttagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcactataatggctggagctttctttgctggattggcatctgatgtccttggctctggacttggttcccttatcaatgctggggctggggccatcaaccaaaaagttgagtttgaaaataacagaaaattgcaacaagcatccttccaatttagcagcaatctacaacaggcttcctttcaacatgacaaagagatgctccaagcacaaattgaggccaccaaaaggctacaacaggaaatgatgaaagttaagcaggcaatgctcctagagggtgggttctctgagacagatgcagcccgcggggcaatcaacgcccccatgacaaaaactttggactggagcgggacaaggtactgggctcccgatgctaggactacaacatacaatgcaggccgcttttccacccctcaaccatcgggggcactgccaggaagagctaatcttagggatgctgtccctgctcggggttcctccagtaagtcttctaattcttctactgccacttctgtgtactcaaatcaaactacttcaacgagacttggttctacagctggttctggtaccagtgtctcgagcttcccgtcaactgcaaggactaggagctgggttgaggatcaaagtaggaatttgtcacctttcatgaggggggcccacaacatatcgtttgtcaccccaccatctagcagatcctctagccaaggcacagtctcaaccgtgcctaaagagattttggactcctggactggcgctttcaacacgcgcaggcagccactcttcgctcacattcgtaagcggggggagtcacgggcgtaatgtgaaaagacaaaattgattatctttcttttctttagtgtctttttaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 399 bp, Human norovirus LM14-2 strain
ggcaagaaagggaagaacaagtccggccgtggcaagaagcacacagccttttcaagtaaagggctcagtgatgaggagtacgatgagtacaagagaatcagagaagaaaggaatggtaagtactccatagaagagtaccttcaggacagagacaggtactacgaggaggtggccattgccagggcaaccgaagaggacttctgtgaagaagaagaggccaaaatccggcagagaatttttagaccaacaaggaaacaacgcaaagaagagagggcctctctcggcttggtcacaggctctgaaatcaggaagagaaacccagaagacttcaaacccaagggaaagctgtgggctgatgacgacagaagtgttgactacaatgagaaactcaactttgag

SEQ ID NO: 14 - protein, 9 aa, Artificial Sequence
  linker
SNSSSVPGG

SEQ ID NO: 15 - protein, 5 aa, Artificial Sequence
  linker
GGGGS

SEQ ID NO: 16 - protein, 5 aa, Artificial Sequence
  linker
PAPAP

SEQ ID NO: 17 - protein, 8 aa, Artificial Sequence
  modified sequence
VEHHHHHH

SEQ ID NO: 18 - DNA, 34 bp, Artificial Sequence
  primer
acgccatatgggcaagaaagggaagaacaagtcc

SEQ ID NO: 19 - DNA, 44 bp, Artificial Sequence
  primer
gctcgaattcgactcaaagttgagtttctcattgtagtcaacac

SEQ ID NO: 20 - DNA, 44 bp, Artificial Sequence
  primer
ccggctccggccccactcgagggaagcaatacaatatttgtacg

SEQ ID NO: 21 - DNA, 32 bp, Artificial Sequence
  primer
ctcaaagttgagtttctcattgtagtcaacac

SEQ ID NO: 22 - DNA, 44 bp, Artificial Sequence
  primer
ggaggcgggggttcactcgagggaagcaatacaatatttgtacg

SEQ ID NO: 23 - protein, 2166 aa, respiratory syncytial virus
MDPIINGSSANVYLTDSYLKGVISFSECNALGSYLFNGPYLKNDYTNLISRQSPLIEHMNLKKLTITQSLISRYHKGELKLEEPTYFQSLLMTYKSMSSSEQIATTNLLKKIIRRAIEISDVKVYAILNKLGLKEKDRVKPNNNSGDENSVLTTIIKDDILSAVENNQSYTNSDKNYSVNQNINIKTTLLKKLMCSMQHPPSWLIHWFNLYTKLNNILTQYRSNEVKSHGFILIDNQTLSGFQFILNQYGCIVYHKGLKKITTTTYNQFLTWKDISLSRLNVCLITWISNCLNTLNKSLGLRCGFNNVVLSQLFLYGDCILKLFHNEGFYIIKEVEGFIMSLILNITEEDQFRKRFYNSMLNNITDAAIKAQKDLLSRVCHTLLDKTVSDNIINGKWIILLSKFLKLIKLAGDNNLNNLSELYFLFRIFGHPMVDERQAMDAVRINCNETKFYLLSSLSTLRGAFIYRIIKGFVNTYNRWPTLRNAIVLPLRWLNYYKLNTYPSLLEITENDLIILSGLRFYREFHLPKKVDLEMIINDKAISPPKDLIWTSFPRNYMPSHIQNYIEHEKLKFSESDRSRRVLEYYLRDNKFNECDLYNCVVNQSYLNNSNHVVSLTGKERELSVGRMFAMQPGMFRQIQILAEKMIAENILQFFPESLTRYGDLELQKILELKAGISNKSNRYNDNYNNYISKCSIITDLSKFNQAFRYETSCVCSDVLDELHGVQSLFSWLHLTIPLVTIICTYRHAPPFIKDHVVNLNEVDEQSGLYRYHMGGIEGWCQKLWTIEAISLLDLISLKGKFSITALINGDNQSIDISKPVRLIEGQTHAQADYLLALNSLKLLYKEYAGIGHKLKGTETYISRDMQFMSKTIQHNGVYYPASIKKVLRVGPWINTILDDFKVSLESIGSLTQELEYRGESLLCSLIFRNIWLYNQIALQLRNHALCNNKLYLDILKVLKHLKTFFNLDSIDTALSLYMNLPMLFGGGDPNLLYRSFYRRTPDFLTEAIVHSVFVLSYYTGHDLQDKLQDLPDDRLNKFLTCVITFDKNPNAEFVTLMRDPQALGSERQAKITSEINRLAVTEVLSIAPNKIFSKSAQHYTTTEIDLNDIMQNIEPTYPHGLRVVYESLPFYKAEKIVNLISGTKSITNILEKTSAIDTTDINRATDMMRKNITLLIRILPLDCNKDKRELLSLENLSITELSKYVRERSWSLSNIVGVTSPSIMFTMDIKYTTSTIASGIIIEKYNVNGLTRGERGPTKPWVGSSTQEKKTMPVYNRQVLTKKQRDQIDLLAKLDWVYASIDNKDEFMEELSTGTLGLSYEKAKKLFPQYLSVNYLHRLTVSSRPCEFPASIPAYRTTNYHFDTSPINHVLTEKYGDEDIDIVFQNCISFGLSLMSVVEQFTNICPNRIILIPKLNEIHLMKPPIFTGDVDIIKLKQVIQKQHMFLPDKISLTQYVELFLSNKALKSGSHINSNLILVHKMSDYFHNAYILSTNLAGHWILIIQLMKDSKGIFEKDWGEGYITDHMFINLNVFFNAYKTYLLCFHRGYGKAKLECDMNTSDLLCVLELIDSSYWKSMSKVFLEQKVIKYIVNQDTSLHRIKGCHSFKLWFLKRLNNAKFTVCPWVVNIDYHPTHMKAILSYIDLVRMGLINVDKLTIKNKNKFNDEFYTSNLFYISYNFSDNTHLLTKQIRIANSELEDNYNKLYHPTPEALENISSIPVKSNNRNKPKFCISGSTESMMTSTFSNKMHIKSSTVTTRFNYSRQDLYNLFPIVVIDRIIDHSGNTEKSNQLYTTTSHQTSLVRNSASLYCMLPWHHVNRFNFVFSSTGCKISIEYILKDLKIKDPSCIAFIGEGAGNLLLRTVVELHPDIRYIYRSLKDCNDHSLPIEFLRLYNGHINIDYGENLTIPATDATNNIHWSYLHIKFAEPISIFVCDAELPVTANWSKIIIEWSKHVRKCKYCSSVNRCILIAKYHAQDDIDFKLDNITILKTYVCLGSKLKGSEVYLVLTIGPANILPVFDVVQNAKLILSRTKNFIMPKKIDKESIDANIKSLIPFLCYPITKNGIKTSLSKLKSVVNGDILSYSIAGRNEVFSNKLINHKHMNILKWLDHVLNFRSAELNYNHLYMIESTYPYLSELLNSLTTNELKKLIKITGSVLYNLPNEQ

SEQ ID NO: 24 - protein, 68 aa, respiratory syncytial virus
KFYLLSSLSTLRGAFIYRIIKGFVNTYNRWPTLRNAIVLPLRWLNYYKLNTYPSLLEITENDLIILSG

SEQ ID NO: 25 - DNA, 35 bp, Artificial Sequence
  primer
ggagatatacatatgaagttctatttattaagtag

SEQ ID NO: 26 - DNA, 36 bp, Artificial Sequence
  primer
tgaacccccgcctcctcctgataaaataatcaaatc

SEQ ID NO: 27 - DNA, 15 bp, Artificial Sequence
  primer
ggaggcgggggttca

SEQ ID NO: 28 - DNA, 21 bp, Artificial Sequence
  primer
atgtatatctccttcttaaag

SEQ ID NO: 29 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 231 aa, SARS-CoV-2
KGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVN

SEQ ID NO: 31 - DNA, 692 bp, SARS-CoV-2
aaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaat

SEQ ID NO: 32 - DNA, 621 bp, SARS-CoV-2
tttcgtgttcagccgaccgaaagcattgttcgttttccgaatatcaccaatctgtgtccgtttggcgaagtttttaatgcaacccgttttgcaagcgtttatgcctggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcatggaatagcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagcctaccaatggtgtgggttatcagccgtatcgtgttgttgttctgtcatttgaactgctgcatgcaccggcaaccgtt

SEQ ID NO: 33 - protein, 208 aa, SARS-CoV-2
MFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 34 - DNA, 15 bp, Artificial Sequence
  primer
ggagatatacatatg

SEQ ID NO: 35 - DNA, 15 bp, Artificial Sequence
  primer
ggaggcgggggttca

SEQ ID NO: 36 - protein, 9 aa, SARS-CoV-2
TLDSKTQSL

SEQ ID NO: 37 - protein, 9 aa, SARS-CoV-2
YLQPRTFLL

SEQ ID NO: 38 - protein, 9 aa, SARS-CoV-2
SPRRARSVA

SEQ ID NO: 39 - protein, 9 aa, SARS-CoV-2
VLNDILSRL

SEQ ID NO: 40 - protein, 9 aa, SARS-CoV-2
SLVKPSFYV

SEQ ID NO: 41 - protein, 9 aa, SARS-CoV-2
KLLEQWNLV

SEQ ID NO: 42 - protein, 9 aa, SARS-CoV-2
SMWSFNPET

SEQ ID NO: 43 - protein, 9 aa, SARS-CoV-2
LPKEITVAT

SEQ ID NO: 44 - protein, 9 aa, SARS-CoV-2
FPRGQGVPI

SEQ ID NO: 45 - protein, 9 aa, SARS-CoV-2
SPRWYFYYL

SEQ ID NO: 46 - protein, 9 aa, SARS-CoV-2
LLLDRLNQL

SEQ ID NO: 47 - protein, 9 aa, SARS-CoV-2
KPRQKRTAT

SEQ ID NO: 48 - protein, 9 aa, SARS-CoV-2
KLDDKDPNF

SEQ ID NO: 49 - protein, 9 aa, SARS-CoV-2
IPIQASLPF

SEQ ID NO: 50 - protein, 9 aa, SARS-CoV-2
ALSKGVHFV

SEQ ID NO: 51 - protein, 9 aa, SARS-CoV-2
APFLYLYAL

SEQ ID NO: 52 - protein, 9 aa, SARS-CoV-2
LLYDANYFL

SEQ ID NO: 53 - protein, 9 aa, SARS-CoV-2
HLVDFQVTI

SEQ ID NO: 54 - protein, 9 aa, SARS-CoV-2
ILFLALITL

SEQ ID NO: 55 - protein, 9 aa, SARS-CoV-2
RARSVSPKL

SEQ ID NO: 56 - protein, 9 aa, SARS-CoV-2
KLFIRQEEV

SEQ ID NO: 57 - protein, 9 aa, SARS-CoV-2
YIDIGNYTV

SEQ ID NO: 58 - protein, 4 aa, Artificial Sequence
  linker
GGGG

(19) United States
(12) Patent Application Publication
(10) Pub. No.: US 2024/0316181 A1
(43) Pub. Date: Sep. 26, 2024

(54) CONJUGATED PROTEIN MONOMER CARRYING PEPTIDE DERIVED FROM PATHOGENIC MICROORGANISM COMPATIBLE WITH MHC MOLECULE, AGGREGATE OF SAID MONOMERS, COMPONENT VACCINE CONTAINING SAID AGGREGATE AS ACTIVE INGREDIENT, AND METHOD FOR ACQUIRING INFORMATION ON SECRETION OF PHYSIOLOGICALLY ACTIVE SUBSTANCE AFTER IMMUNIZATION

(71) Applicants: TOKYO INSTITUTE OF TECHNOLOGY, Tokyo (JP); THE KITASATO INSTITUTE, Tokyo (JP)

(72) Inventors: Takafumi UENO, Tokyo (JP); Dan Que NGUYEN, Tokyo (JP); Kazuhiko KATAYAMA, Tokyo (JP); Reiko TODAKA, Tokyo (JP); Kei HAGA, Tokyo (JP); Akihito SAWADA, Tokyo (JP)

(73) Assignees: TOKYO INSTITUTE OF TECHNOLOGY, Tokyo (JP); THE KITASATO INSTITUTE, Tokyo (JP)

(21) Appl. No.: 18/271,078

(22) PCT Filed: Jan. 7, 2022
(86) PCT No.: PCT/JP2022/000327

§ 371 Date: Feb. 8, 2024

(30) Foreign Application Priority Data
Jan. 7, 2021 (JP) 2021-001522

(51) Int. Cl. A61K 39/215; A61K 39/12; A61P 37/04; C07K 14/005; G01N 33/50

(52) U.S. Cl. CPC A61K 39/215; A61K 39/12; A61P 37/04; C07K 14/005; G01N 33/5091; C12N 2760/18534; C12N 2770/20034

(57) ABSTRACT

An object of the present invention is to establish means for providing a component vaccine which can selectively or intensively induce cell-mediated immunity mainly attributable to MHC class I, and humoral immunity mainly attributable to MHC class II. The inventors have found that the object can be attained by providing a component vaccine containing, as an active ingredient, a trimer and/or a hexamer of a molecular needle carrying a peptide binding to MHC class I and/or a peptide binding to MHC class II. The inventors have also found an information acquisition method that can determine the MHC class or the like of a test peptide or a similar substance by detecting a change in secretion of a physiologically active substance such as a cytokine in a test animal which has been infected with a target microorganism.

### TECHNICAL FIELD

The present invention relates to a functional protein and to a vaccine employing the protein. More particularly, the invention relates to a “composite protein carrying a peptide which is compatible with an MHC (major histocompatibility complex) molecule and which originates from a pathogenic microorganism such as a virus or a bacterium” (i.e., a monomer), the composite protein serving as an immunogen; to an associated product of the monomer (i.e., a molecular needle); and to a component vaccine containing the associated product as an active ingredient (i.e., an infection-protective antigen). Furthermore, the invention relates to a method for acquiring information about secretion of a physiologically active substance after immunization with the peptide or protein serving as an active ingredient of the vaccine.

### BACKGROUND ART

There has been provided a technique called “molecular needle,” which has been developed by focusing on the excellent function of a bacteriophage to introduce genes into a cell (see Patent Document 1).

Previously, the present inventors invented a composite protein containing a structural protein of norovirus attached to a molecular needle to thereby provide a component vaccine against norovirus. The inventors filed a patent application for the invention (see Patent Document 2).

### PRIOR ART DOCUMENTS

### Patent Documents

Patent Document 1: Japanese Patent Application Laid-Open (kokai) No. 2015-163056
Patent Document 2: WO 2018/074558, pamphlet Non-Patent Documents
Non-patent Document 1: Hoffmann et al., 2020, Cell 181, 1-10 Apr. 16, 2020
Non-patent Document 2: Daniel Wrapp et al., Science, published online February 2020, science abb2507

### DISCLOSURE OF THE INVENTION

### Problems to be Solved by the Invention

In the development of vaccines, “disease enhancement” is a considerably serious problem. “Disease enhancement” is such a phenomenon that inflammation caused upon or after subsequent infection is aggravated by an antibody acquired by infection prophylactic vaccination or another factor. Currently, the generation mechanism of “disease enhancement” has not been precisely elucidated. A case of disease enhancement which may possibly be caused by COVID-19 (SARS-CoV-2) vaccination is called antibody-dependent enhancement (ADE) or enhanced respiratory disease (ERD). In the development of infection prophylactic vaccines against β-coronavirus, possible occurrence of infection enhancement has been reported in animal bioassay of infection prophylactic vaccines against severe acute respiratory syndrome (SARS) virus and middle east respiratory syndrome (MERS) virus (Honda-Okubo Y, et al., J Virol. 2015; 89(6): 2995-3007, Agrawal A S, et al., Hum Vaccin Immunother. 2016; 12(9): 2351-56). Other than the cases of coronavirus vaccines, deaths and aggravation cases which may conceivably be caused by disease enhancement have been reported in clinical trials of infection prophylactic vaccines against RS virus, dengue virus, etc. (Castilow E M, et al., Immunol Res. 2007; 39 (1-3): 225-39, Halstead S B. Hum Vaccin Immunother. 2018; 14 (9): 2158-62).

Thus, disease enhancement involved in vaccination is an obstacle in the development of vaccines.

### Means for Solving the Problems

The present inventors have conducted extensive studies so as to solve the aforementioned problems by use of a component vaccine containing a molecular needle. Surprisingly, the inventors have found that using, as an active ingredient, a molecular needle carrying a peptide (i.e., to which a peptide has been attached) which is compatible with an MHC molecule and which originates from a pathogenic microorganism can realize choice of cell-mediated immunity or humoral immunity to be induced by vaccination; modulation of the balance between two immunity types; and efficient enhancement in protective effect against a pathogenic microorganism. The present invention has been accomplished on the basis of this finding. According to the present invention, problematic “disease enhancement involved in vaccination” can be avoided. Also, target immunity induction can be achieved extremely efficiently and accurately. In addition, by using it as a booster vaccine, it is possible to selectively enhance necessary immune response from the memory immunity induced by a primary vaccine.

The vaccine of the present invention is a component vaccine containing, as an active ingredient, a molecular needle carrying a peptide which is compatible with MHC class I and which originates from a pathogenic microorganism and/or a peptide which is compatible with MHC class II and which originates from the pathogenic microorganism.

That is, the peptide carried by the aforementioned molecular needle (i.e., an active ingredient of the vaccine of the present invention) includes the following three modes: (1) a peptide which is compatible with MHC class I and which originates from a pathogenic microorganism; (2) a peptide which is compatible with MHC class II and which originates from a pathogenic microorganism; and (3) a peptide which is compatible with MHC class I and which originates from a pathogenic microorganism and a peptide which is compatible with MHC class II and which originates from the pathogenic microorganism.

In other words, the present invention provides a first vaccine, which is a component vaccine containing, as an active ingredient, a molecular needle carrying a peptide which is compatible with MHC class I and which originates from a pathogenic microorganism (i.e., the vaccine 1 of the present invention); a second vaccine, which is a component vaccine containing, as an active ingredient, a molecular needle carrying a peptide which is compatible with MHC class II and which originates from a pathogenic microorganism (i.e., the vaccine 2 of the present invention); and a third vaccine, which is a component vaccine containing, as an active ingredient, a molecular needle carrying a peptide which acts as both of a peptide which is compatible with MHC class I and which originates from a pathogenic microorganism and a peptide which is compatible with MHC class II and which originates from the pathogenic microorganism (i.e., the vaccine 3 of the present invention).

The expression “which is compatible with MHC class I” refers to an action of promoting a specific function of an MHC class I molecule in the immune system of a living body (described later; briefly, induction of cell-mediated immunity), and the expression “which is compatible with MHC class II” refers to an action of promoting a specific function of an MHC class II molecule in the immune system of a living body (described later; briefly, preferential induction of humoral immunity).

The vaccine 1 of the present invention can selectively induce cell-mediated immunity against a target pathogenic microorganism. The vaccine 2 of the present invention can preferentially induce humoral immunity against a target pathogenic microorganism. Even when the vaccines 1 and 2 of the present invention target the same pathogenic microorganism, they may be used in combination or singly. By appropriately selecting the mode of use (i.e., combination or single), the balance between induced cell-mediated immunity and humoral immunity can be achieved, or the protective effect against a pathogenic microorganism can be efficiently enhanced. For example, disease enhancement involved in vaccination is thought to be caused by uncontrolled humoral immunity in general, and the disease enhancement can be prevented by use of only the vaccine 1 of the present invention, or by use of the vaccines 1 and 2 of the present invention at a reduced vaccine 2 ratio. In contrast, disease enhancement caused by uncontrolled cell-mediated immunity is also known. In this case, in contrast to the above case, the vaccine 2 of the present invention, which contains a molecular needle carrying an MHC class II peptide as an active ingredient, is preferably used. Also, it is possible to efficiently design a vaccine exhibiting high protective effect on a pathogenic microorganism by selecting the associated products 1 and 2 of the present invention so as to actively exert both cell-mediated immunity and humoral immunity and using them in combination as active ingredients of the vaccine. The vaccine 3 of the present invention contains, as an active ingredient, a molecular needle carrying a peptide including, in a predetermined combination and ratio, a peptide which is compatible with MHC class I and which originates from a pathogenic microorganism and a peptide which is compatible with MHC class II and which originates from the pathogenic microorganism. Alternatively, the vaccine 3 of the present invention contains, as an active ingredient, a molecular needle carrying a peptide which acts as both of a peptide which is compatible with MHC class I and which originates from a pathogenic microorganism and a peptide which is compatible with MHC class II and which originates from the pathogenic microorganism (particular preferred examples of such a peptide with acts as both of the peptides include a peptide sequence which binds to a B cell receptor in a humoral immunity pathway involving MHC class II). The vaccine 3 of the present invention is a component vaccine which models the balance between cell-mediated immunity and humoral immunity, or effectively exerts both cell-mediated immunity and humoral immunity, through employment of such a molecular needle as an active ingredient.

As described above, the vaccine of the present invention conceptually includes the vaccines 1, 2, and 3 of the present invention.

The molecular needle serving as an active ingredient of the vaccine 1 of the present invention is an associated product of the below-described composite protein 1 of the present invention (associated product 1 of the present invention). The molecular needle serving as an active ingredient of the vaccine 2 of the present invention is an associated product of the below-described composite protein 2 of the present invention (associated product 2 of the present invention). The molecular needle serving as an active ingredient of the vaccine 3 of the present invention is an associated product of the below-described composite protein 3 of the present invention (associated product 3 of the present invention).

### [1] Composite Protein of the Present Invention

The composite protein is a composite protein presented by the following amino acid sequence (1):

$$W - {L}_{1} - {X}_{n} - Y \qquad ( 1 )$$

[wherein W represents one or more amino acid sequences of a peptide including one or more peptides selected from peptides which are compatible with MHC class I and which originate from a pathogenic microorganism and/or peptides which are compatible with MHC class II and which originate from the pathogenic microorganism, serving as an immunogen; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):

$${Y}_{1} - {L}_{2} - {Y}_{2} - {Y}_{3} \qquad ( 2 )$$

[wherein Y₁ represents any one amino acid sequence selected from the group consisting of SEQ ID NOS: 2 to 5; Y₂ represents any one amino acid sequence selected from the group consisting of SEQ ID NOS: 6 to 9; L₂ represents a second linker sequence having 0 to 30 amino acids; Y₃ represents an amino acid sequence for modification; and either of Y₂ and Y₃ may be absent]. The amino acid sequence represented by Xₙ, Y₁, or Y₂ may include a modified amino acid sequence thereof obtained by deleting, substituting, or adding one or more amino acid residues from, in, or to the original amino acid sequence.

Notably, in formulas (1) and (2), the symbol “-” connecting the amino acid sequence units denotes a simple molecular bond (substantially a peptide bond) which is used to clearly isolate from one another the amino acid sequences each having a specific meaning (e.g., W, L₁, Xₙ, and Y).

In the amino acid sequence represented by Xₙ, Y₁, or Y₂, the term “deleting” refers to deletion of any amino acid residue in the amino acid sequence included in the above formula and represented by any of the SEQ ID NOS. The amino acid residue at the N-terminal side of the deleted amino acid residue and that at the C-terminal side of the deleted amino acid residue are linked via a peptide bond. In the case of deletion of the N-terminal amino acid residue or the C-terminal amino acid residue, no linkage is present. The number of deleted residues is counted as “the number of amino acid deletions.” The term “substituting” refers to substitution of any amino acid residue in the amino acid sequence included in formula (1) and represented by any of the SEQ ID NOS, “with another amino acid residue.” The new amino acid residue is linked to the amino acid residue at the N-terminal side thereof and at the C-terminal side thereof via a peptide bond. In the case of substitution of the N-terminal side or C-terminal side amino acid residue, the amino acid residue is linked via a peptide bond to another C-terminal side or N-terminal side amino acid residue. The number of substituted residues is counted as “the number of amino acid substitutions.” The term “adding” refers to addition of one or more new amino acid residues to one or more peptide bond sites in the amino acid sequence included in formula (1) and represented by any of the SEQ ID NOs, to thereby form a new peptide bond (s). The type and number of the above modifications of amino acid residue may be elucidated by comparison in alignment of the amino acid sequence represented by formula (1) with the resultant amino acid sequence through manpower or employment of appropriate analysis software.

The linker sequence L₁ or L₂, or the modification amino acid sequence Y₃ may have any desired sequence within the range of the number of amino acid residues as defined in the above formula.

Further, the trimer or hexamer of the modified composite protein having the modified amino acid sequence preferably has substantially the same immunostimulation activity as that of the trimer or hexamer of the composite protein of the above formula. The expression “substantially the same” refers to such a similarity that no significant difference in immunostimulation activity between the trimer or hexamer of a composite protein having the non-modified amino acid sequence and that of a composite protein having the modified amino acid sequence is confirmed within a significance level range of 5%, when the immunostimulation activity is determined through an established technique such as the “neutralization test.”

In the modified amino acid sequence obtained through deletion, substitution, or addition of one or more amino acid residues from, in, or to the amino acid sequence represented by Xₙ, Y₁, or Y₂ included in the above formula, the number of modifications of amino acid residues in each amino acid sequence is preferably ≤8n, more preferably ≤4n, still more preferably ≤2n, most preferably ≤1n in the case of Xₙ; preferably ≤30, more preferably ≤20, still more preferably ≤10, considerably preferably ≤2, most preferably ≤1 in the case of Y₁; and preferably ≤15, more preferably ≤10, still more preferably ≤5, considerably preferably ≤2, most preferably ≤1 in the case of Y₂. The deletion, substitution, or addition of these amino acids is acceptable, only when the composite proteins 1, 2, and 3 of the present invention each form a trimer or a hexamer thereof.

Among the composite proteins falling within the scope of the present invention, the composite protein 1 of the present invention corresponds to an embodiment in which W contains, as an immunogen, only a peptide which is compatible with MHC class I and which originates from a pathogenic microorganism; the composite protein 2 of the present invention corresponds to an embodiment in which W contains, as an immunogen, only a peptide which is compatible with MHC class II and which originates from a pathogenic microorganism; and the composite protein 3 of the present invention corresponds to an embodiment in which W contains, as an immunogen, both a peptide which is compatible with MHC class I and which originates from a pathogenic microorganism, and a peptide which is compatible with MHC class II and which originates from the pathogenic microorganism.

In the composite protein of the present invention, a preferred embodiment of the immunogen W is a peptide in which two or more peptides selected from peptides which are compatible with MHC class I and which originate from a pathogenic microorganism and/or peptides which are compatible with MHC class II and which originate from the pathogenic microorganism are linked via a linker. Also, as shown in the Examples, another preferred embodiment of W is a peptide containing both a peptide segment (motif) which is compatible with MHC class I and a peptide segment (motif) which is compatible with MHC class II. Still another preferred embodiment of W including an MHC class II motif is a peptide which can be recognized by a B cell receptor and which originates from a pathogenic microorganism.

The present invention also provides a vector for gene expression into which a nucleic acid fragment encoding the composite protein of the present invention has been incorporated (hereinafter may also be referred to as the vector of the present invention), and a transformant which has been transformed by a nucleic acid fragment encoding the composite protein of the present invention (hereinafter may also be referred to as the transformant of the present invention). The vector of the present invention includes the following three embodiments: a vector in which a nucleic acid fragment encoding the composite protein 1 of the present invention has been incorporated (hereinafter may be referred to as the vector 1 of the present invention); a vector in which a nucleic acid fragment encoding the composite protein 2 of the present invention has been incorporated (hereinafter may be referred to as the vector 2 of the present invention); and a vector in which a nucleic acid fragment encoding the composite protein 3 of the present invention has been incorporated (hereinafter may be referred to as the vector 3 of the present invention).

The transformant of the present invention can be easily produced by transforming a host with the vector of the present invention. The transformant of the present invention includes the following three embodiments: a transformant transformed by the vector 1 of the present invention (i.e., the transformant 1 of the present invention); a transformant transformed by the vector 2 of the present invention (i.e., the transformant 2 of the present invention); and a transformant transformed by the vector 3 of the present invention (i.e., the transformant 3 of the present invention).

By performing gene expression in the transformant of the present invention, the composite protein of the present invention can be produced.

### [2] Associated Product of the Present Invention

### (2-1) Associated Product 1 of the Present Invention

The associated product 1 of the present invention, which serves as an active ingredient of the vaccine 1 of the present invention, is formed of the composite protein 1 of the present invention as a monomer. More specifically, the associated product 1 includes a trimer or a hexamer of the composite protein 1, or a mixture of the trimer and the hexamer. Hereinafter, the trimer or the hexamer of the composite protein 1 of the present invention and the mixture of the trimer and the hexamer may be collectively referred to simply as a “trimer and/or hexamer 1.” In other words, the associated product 1 of the present invention contains a trimer and/or hexamer of the composite protein 1 of the present invention as a monomer. Furthermore, in view of the below-mentioned procedure of producing the associated product 1 of the present invention, the associated product 1 may be defined as an associated product formed through association of molecules of the composite protein 1 of the present invention in an aqueous liquid. The associated product 1 of the present invention, per se, can penetrate the target cells.

The aforementioned trimer is a trimeric protein formed of the composite protein 1 of the present invention as a monomer, wherein the monomer units are composite proteins of the present invention which are identical to or different from one another, and the aforementioned hexamer is a hexameric protein formed through association of two molecules of the trimeric protein 1.

The associated product 1 of the present invention may be produced by bringing the molecules of the composite protein 1 of the present invention into contact with one another in an aqueous liquid. In some conceivable cases, the associated product 1 of the present invention may fail to be formed depending on the compatibility between the attached peptides, or the formed associated product may fail to be soluble in aqueous liquid. However, by bringing the molecules of the target composite protein 1 into contact with one another in an aqueous liquid, and confirming the results through SDS-PAGE, high-speed atomic force microscopy, gel filtration chromatography, or the like, it is possible to easily evaluate whether the target composite protein forms a trimer or a hexamer thereof, and whether or not the product can be used as an active ingredient of the vaccine 1 of the present invention. Furthermore, solubility of the associated product in aqueous liquid can be readily determined by actually conducting a solubilization test.

### (2-2) Associated Product 2 of the Present Invention

The associated product 2 of the present invention, which serves as an active ingredient of the vaccine 2 of the present invention, is formed of the composite protein 2 of the present invention as a monomer. More specifically, the associated product 2 includes a trimer or a hexamer of the composite protein 2, or a mixture of the trimer and the hexamer. Hereinafter, the trimer or the hexamer of the composite protein 2 of the present invention and the mixture of the trimer and the hexamer may be collectively referred to simply as a “trimer and/or hexamer 2.” In other words, the associated product 2 of the present invention contains a trimer and/or hexamer of the composite protein 2 of the present invention as a monomer. Furthermore, in view of the below-mentioned procedure of producing the associated product 2 of the present invention, the associated product 2 may be defined as an associated product formed through association of molecules of the composite protein 2 of the present invention in an aqueous liquid. The associated product 2 of the present invention, per se, can penetrate the target cells.

The aforementioned trimer is a trimeric protein formed of the composite protein 2 of the present invention as a monomer, wherein the monomer units are composite proteins of the present invention which are identical to or different from one another, and the aforementioned hexamer is a hexameric protein formed through association of two molecules of the trimeric protein 2.

The associated product 2 of the present invention may be produced by bringing the molecules of the composite protein 2 of the present invention into contact with one another in an aqueous liquid. In some conceivable cases, the associated product 2 of the present invention may fail to be formed depending on the compatibility between the attached peptide, or the formed associated product may fail to be soluble in an aqueous liquid. However, by bringing the molecules of the target composite protein 2 into contact with one another in an aqueous liquid, and confirming the results through SDS-PAGE, high-speed atomic force microscopy, gel filtration chromatography, or the like, it is possible to easily evaluate whether the target composite protein forms a trimer or a hexamer thereof, and whether or not the product can be used as an active ingredient of the vaccine 2 of the present invention. Furthermore, solubility of the associated product in aqueous liquid can be readily determined by actually conducting a solubilization test.

### (2-3) Associated Product 3 of the Present Invention

The associated product 3 of the present invention, which serves as an active ingredient of the vaccine 3 of the present invention, is formed of the composite protein 3 of the present invention as a monomer. More specifically, the associated product 3 includes a trimer and/or a hexamer of the composite protein 3, or a mixture of the trimer and the hexamer. Hereinafter, the trimer or the hexamer of the composite protein 3 of the present invention and the mixture of the trimer and the hexamer may be collectively referred to simply as a “trimer and/or hexamer 3.” In other words, the associated product 3 of the present invention contains a trimer and/or hexamer of the composite protein 3 of the present invention as a monomer. Furthermore, in view of the below-mentioned procedure of producing the associated product 3 of the present invention, the associated product 3 may be defined as an associated product formed through association of molecules of the composite protein 3 of the present invention in an aqueous liquid. The associated product 3 of the present invention, per se, can penetrate the target cells.

The aforementioned trimer is a trimeric protein formed of the composite protein 3 of the present invention as a monomer, wherein the monomer units are composite proteins of the present invention which are identical to or different from one another, and the aforementioned hexamer is a hexameric protein formed through association of two molecules of the trimeric protein 3.

The associated product 3 of the present invention may be produced by bringing the molecules of the composite protein 3 of the present invention into contact with one another in an aqueous liquid. In some conceivable cases, the associated product 3 of the present invention may fail to be formed depending on the compatibility between the attached peptides. In such a case, the target composite protein 3 is caused to be in contact with a molecular needle in an aqueous liquid, or the formed associated product may fail to be soluble in aqueous liquid. However, by bringing the molecules of the target composite protein 3 into contact with one another in an aqueous liquid, and confirming the results through SDS-PAGE, high-speed atomic force microscopy, gel filtration chromatography, or the like, it is possible to easily evaluate whether the target composite protein forms a trimer or a hexamer thereof, and whether or not the product can be used as an active ingredient of the vaccine 3 of the present invention. Furthermore, solubility of the associated product in aqueous liquid can be readily determined by actually conducting a solubilization test.

### [3] The Vaccine of the Present Invention

### (3-1) The Vaccine 1 of the Present Invention

The vaccine 1 of the present invention contains the associated product 1 of the present invention as an active ingredient (infection-protective antigen). More specifically, the vaccine 1 is a component vaccine for inducing cell-mediated immunity, the vaccine containing, as an active ingredient, one or more species of a peptide including one or more peptides which are compatible with MHC class I and which originate from a pathogenic microorganism. The vaccine is suitably administered in a transmucosal, percutaneous, subcutaneous, intradermal, or intramuscular manner.

In other words, the vaccine 1 of the present invention is a component vaccine in relation to “use of the composite protein 1 of the present invention or the associated product 1 of the present invention, wherein the composite protein 1 is associated to form the associated product 1, and the associated product is used as an active ingredient in the vaccine 1 of the present invention to activate cell-mediated immunity.”

In order to further enhance immunogenicity, the vaccine 1 of the present invention may contain an adjuvant, for example, a molecular needle carrying B subunit of cholera toxin as W. However, the vaccine 1 of the present invention may be an adjuvant-free (i.e., adjuvant-removed) vaccine. The adjuvant-free vaccine is rather preferred, from the viewpoint of attaining an advantageous feature of the vaccine 1 of the present invention.

No particular limitation is imposed on the animal to which the vaccine 1 of the present invention can be administered. The vaccine is applicable to all animals, including humans, which may be infected with a specific pathogenic microorganism. Examples of the target subject include dog and cat.

### (3-2) The Vaccine 2 of the Present Invention

The vaccine 2 of the present invention contains the associated product 2 of the present invention as an active ingredient (infection-protective antigen). More specifically, the vaccine 2 is a component vaccine for inducing humoral immunity, the vaccine containing, as an active ingredient, one or more species of a peptide including one or more peptides which are compatible with MHC class II and which originate from a pathogenic microorganism. The vaccine is suitably administered in a transmucosal, percutaneous, subcutaneous, intradermal, or intramuscular manner.

In other words, the vaccine 2 of the present invention is a component vaccine in relation to “use of the composite protein 2 of the present invention or the associated product 2 of the present invention, wherein the composite protein 1 is associated to form the associated product 1, and the associated product is used as an active ingredient in the vaccine 2 of the present invention to preferentially activate humoral immunity.”

In order to further enhance immunogenicity, the vaccine 2 of the present invention may contain an adjuvant, for example, a molecular needle carrying B subunit of cholera toxin as W. However, the vaccine 2 of the present invention may be an adjuvant-free (i.e., adjuvant-removed) vaccine. The adjuvant-free vaccine is rather preferred, from the viewpoint of attaining an advantageous feature of the vaccine 2 of the present invention.

No particular limitation is imposed on the animal to which the vaccine 2 of the present invention can be administered. The vaccine is applicable to all animals, including humans, which may be infected with a specific pathogenic microorganism. Examples of the target subject include dog and cat.

### (3-3) Mixed Mode of the Vaccines 1 and 2 of the Present Invention

The vaccines 1 and 2 of the present invention may be used singly or in combination. In other words, the associated products 1 and 2 of the present invention may be mixed to provide the active ingredient of the vaccine of the present invention. In this case, the ratio of the associated product 1 of the present invention to the associated product 2 of the present invention may be determined in accordance with the type of the pathogenic microorganism (target for infection prevention) and the subject of vaccination. In the case where a pathogenic microorganism may cause disease enhancement after inoculation of a conventional vaccine as described above, it is possible to verify whether the disease enhancement is attributed to cell-mediated immunity or humoral immunity, and, based on the verification results, to select the ratio of the vaccine 1 of the present invention to the vaccine 2 of the present invention. For example, in the case where the results show that the disease enhancement is caused by humoral immunity, preferably, the ratio of the associated product 2 of the present invention, which preferentially induces humoral immunity, is reduced, and the ratio of the associated product 1 of the present invention is increased, to thereby provide a vaccine which predominantly induces cell-mediated immunity.

### (3-4) The Vaccine 3 of the Present Invention

The vaccine 3 of the present invention contains the associated product 3 of the present invention as an active ingredient (infection-protective antigen). More specifically, the vaccine 3 is a component vaccine for inducing both cell-mediated immunity and humoral immunity, the vaccine containing, as an active ingredient, one or more species of a peptide including one or more peptides which are compatible with MHC class I and which originate from a pathogenic microorganism, and one or more peptides which are compatible with MHC class II and which originate from the pathogenic microorganism. The vaccine is suitably administered in a transmucosal, percutaneous, subcutaneous, intradermal, or intramuscular manner.

In other words, the vaccine 3 of the present invention is a component vaccine in relation to “use of the composite protein 3 of the present invention or the associated product 3 of the present invention, wherein the composite protein 3 is associated to form the associated product 3, and the associated product is used as an active ingredient in the vaccine 3 of the present invention to activate both cell-mediated immunity and humoral immunity.”

In order to further enhance immunogenicity, the vaccine 3 of the present invention may contain an adjuvant, for example, a molecular needle carrying B subunit of cholera toxin as W. However, the vaccine 3 of the present invention may be an adjuvant-free (i.e., adjuvant-removed) vaccine. The adjuvant-free vaccine is rather preferred, from the viewpoint of attaining an advantageous feature of the vaccine 3 of the present invention.

No particular limitation is imposed on the animal to which the vaccine 3 of the present invention can be administered. The vaccine is applicable to all animals, including humans, which may be infected with a specific pathogenic microorganism. Examples of the target subject include dog and cat.

### [4] Associated Product Production Method of the Present Invention

The associated product of the present invention is produced through the following procedure. Specifically, 3 or more molecules of the composite protein of the present invention are brought into contact with one another by the mediation of aqueous liquid, to thereby associate the composite protein molecules as monomers and to form a mixture of a trimer and a hexamer. If needed, the trimer or the hexamer may be selectively isolated and recovered.

The composite protein of the present invention per se can be produced by expressing a nucleic acid fragment encoding the target composite protein through a genetic engineering procedure. Alternatively, the composite protein may be synthesized through a technique of peptide synthesis. When the composite protein molecules are in contact with one another in aqueous liquid, a trimer and a hexamer of the composite protein are formed in a spontaneous manner. As a result, a mixture containing a trimer and a hexamer of the composite protein is provided. Through selectively isolating and recovering the trimer and the hexamer, the trimer and the hexamer of interest can be separately yielded.

The “aqueous liquid” will next be described. Particularly when the composite protein of the present invention is produced through a genetic engineering procedure, the composite protein is biologically formed through gene expression. Thereafter, in one procedure, cells in which the target composite protein has been formed are collected, and broken or lysed to extract the composite protein, and the target composite protein is isolated through a known isolation method. These steps are performed in an aqueous liquid such as water or a buffer. In the aqueous liquid, association proceeds spontaneously, to thereby yield a mixture of the trimer and the hexamer, which are the associated products of the present invention. Alternatively, when the composite protein of the present invention is produced through total chemical synthesis, or part-to-part individual synthesis, followed by combining the parts through chemical modification, the product is suspended in an aqueous liquid such as water or a buffer. In this case, association also proceeds spontaneously, to thereby yield a mixture of the trimer and the hexamer, which are the associated products of the present invention.

No particular limitation is imposed on the method of isolating and recovering the target trimer and hexamer from the mixture containing the target trimer and hexamer, and a known molecular-weight-basis separation method may be employed. Examples of the separation method include gel electrophoresis, affinity chromatography, molecular sieve separation (e.g., size exclusion chromatography), and ion-exchange chromatography.

Thus, one most preferred mode of the associated product production method of the present invention is “a production method including culturing, in a liquid culture medium, a transformant into which a nucleic acid fragment encoding the composite protein of the present invention has been incorporated, to thereby form the corresponding composite protein through gene expression; and obtaining a mixture containing a trimer and a hexamer of the composite protein (as a monomer), the mixture being formed via a spontaneous association process, or the production method further including selectively isolating and recovering the trimer or the hexamer from the mixture.”

The thus-produced associated product of the present invention may be used as the active ingredient of the vaccine of the present invention.

[5] Pathogenic Microorganism to which the Present Invention can be Applied

In the case where a peptide which is compatible with MHC class I or class II is used as the aforementioned W, examples of the pathogenic microorganism which can be used in this case include a virus, a bacterium, a fungus, and a protozoa.

The virus may be a DNA virus, an RNA virus, or a retrovirus. Examples of the pathogenic (infectious) virus to which a certain vaccine has already been provided include poliovirus, measles virus, rubella virus, mumps virus, chickenpox virus, yellow fever virus, rotavirus, herpes zoster virus (herpesvirus), influenza virus, norovirus, astrovirus, sapovirus, rabies virus, Japanese encephalitis virus, hepatitis viruses (e.g., HAV, HBV, and HCV), human papilloma virus, HIV, and ebolavirus. Examples of the pathogenic (infectious) virus to which no vaccine has been provided include RS virus (RSV), Dengue virus, and coronavirus (including a new coronavius). One characteristic feature of the vaccine of the present invention resides in that the vaccine may be effective for the infectious virus to which no vaccine has been provided. Regarding these viruses to which no vaccine has been provided, disease enhancement (cytokine storm) is reported to occur after vaccination. The disease enhancement is an obstacle to the development of such vaccines. The present invention can be applied to such viruses that have a risk of disease enhancement after vaccination.

Examples of the bacterium include Pneumococcus, Mycobacterium tuberculosis, Vibrio cholerae, Bacillus dysenteriae, Bacillus typhosus, Bacillus anthracis, Neisseria meningitidis, Clostridium tetani, Corynebacterium diphtheriae, and Bordetella pertussis.

Examples of the fungus include Trichophyton, Candida, Cryptococcus, and Aspergillus.

Examples of the protozoa include Plasmodium.

### [6] Peptide Carried (W)

(1) Peptide which is Compatible with MHC Class I (Hereinafter Also Referred to as “Class I Peptide”)

The class I peptide contains an essential region formed of usually 8 to 10 amino acid residues. The class I peptide includes a plurality of short peptides characteristic to each target pathogenic microorganism. The peptide including both ends links to a MHC class I molecule in a peptide-binding groove specific to each class I peptide via several anchor residues. Even when the peptide has more than 10 amino acid residues, if the C-terminal is linkable to an MHC class I molecule, the peptide can link to an MHC class I molecule. In such a case, the peptide is cut and shortened by an exopeptidase in the endoplasmic reticulums after linking. The thus-formed peptide-MHC class I complex is presented on the cell surfaces, and the thus-presented peptide-MHC class I complex is recognized by “T cells (typically, CD8⁺ T cells) which are primarily stimulated by the peptide-MHC class I complex and which specifically attacks to the complex.” This recognition mechanism serves as a signal for cytotoxic CD8⁺ T cell to attack cells infected with a target pathogenic microorganism and presented with the class I peptide. As a result, cell-mediated immunity with respect to the target pathogenic microorganism is induced.

Examples of the cytokine or chemokine which is released from immunocompetent cells upon induction of cell-mediated immunity by the class I peptide include I-type IFM, which is released at the first stimulation, and also, IFN-γ, IL-2, IL-12, MIP-1a, MIP-1b, TNF-α, IL-10, perforin, granzyme a, granzyme b, and RANTES, which are released upon differentiation into CTL and furthermore upon cell damege by CTL. By detecting these cytokines or chemokines, cell-mediated immunity attributed to MHC class I can be detected.

Each terminal of the class I peptide is fixed to an MHC class I molecule by bonding between N-terminal and C-terminal atoms of the class I peptide having the aforementioned number of amino acid residues and atoms forming well-conserved amino acid residues (e.g., a cluster of tyrosine residues) at each end of the peptide-binding groove of the MHC class I molecule. Meanwhile, the main difference between different MHC class I alleles resides in the code of a specific site of the peptide-binding groove. As a result, by virtue of the difference in amino acids forming the site, different MHC class I molecules preferentially bind to respective compatible class I peptides. The class I peptide which binds to a specific MHC class I molecule has completely the same or almost the same amino acid residues at two or three positions along the amino acid sequence. By the mediation of bonding with specific amino acid side chains present in the binding groove of an MHC class I molecule, which chains are compatible with the side chains of the amino acid residues present at the above positions of the class I peptide, the class I peptide is fitted in the binding groove of the MHC class I molecule. Such a particular amino acid residue of the class I peptide is called “an anchor residue.” The position and character of each anchor residue vary among class I peptides. However, most class I peptides have a hydrophobic (or basic) anchor residue at the C terminal. Examples of the hydrophobic amino acid include aromatic amino acids such as phenylalanine and tyrosine; and hydrophobic amino acids such as valine, leucine, and isoleucine. Examples of the basic amino acid include lysine, arginine, and histidine. Notably, a peptide having a suitable length including these anchor residues does not always bind to the MHC class I molecule, and the binding also depends on the properties of amino acid residues of other positions of the peptide. Such an amino acid residue is called “a secondary anchor.”

In selection of the class I peptide, a candidate peptide sequence is selected from the nucleotide sequence of the gene of a target pathogenic microorganism or the amino acid sequences encoded by it by use of a computer program including an algorithm for selecting characteristics of the class I peptide, or a related database (e.g., http://tools.iedb.org/main/; http://tools.immuneepitope.org/main/datasets/; or Genetyx software, T cell epitope prediction), and the corresponding peptide is synthesized. The thus-synthesized peptide is tested in terms of activation of cell-mediated immunity as an index, whereby a class I peptide of interest is obtained. Examples of suitable test methods include ELISpot assay. In ELISpot assay, firstly, cells (cells for infection) are fixed in wells of a multi-well plate, and the cells are transfected with a candidate class I peptide. No particular limitation is imposed on the transfection method, and examples of the method include introduction of a plasmid in which a candidate class I peptide has been incorporated by the mediation of a lipoprotein vehicle, or an RNA fragment including a portion encoding the candidate class I peptide. Thus, in the cells transfected with the candidate class I peptide, if the candidate is actually a class I peptide, the peptide binds to MHC class I molecules of the test cells, and is present on the surfaces of the cells. Subsequently, the thus-obtained cells are caused to react with leucocytes from a mammal such as a human cured of the target disease (typically CD8⁺ T cells). Since the leucocytes attack to the infection-target cells on which class I peptide-bound MHC class I molecules are present, the target cells in the wells containing a class I peptide are lysed to form plaques. The test peptide which can form such plaques is evaluated as a class I peptide of interest. Through the above procedure, the class I peptide of interest can be characterized and obtained.

Alternatively, as described above, selection of the class I peptide can be achieved by detecting a cytokine or chemokine which is characteristic of function of cell-mediated immunity induced by MHC class I. The detection may be performed by use of a molecular needle carrying the test peptide as shown in the Examples. Details of the detection will be described below.

(2) Peptide which is Compatible with MHC Class II (Hereinafter Also Referred to as “Class II Peptide”)

The class II peptide contains about 8 or more essential amino acid residues, and has different lengths and characteristic of each target pathogenic microorganism. The class II peptide links to a MHC class II molecule in a peptide-binding groove specific to each class II peptide via several anchor residues, to thereby form a peptide-MHC class II molecule complex. Differing from MHC class I, both ends of the class II peptide do not link to an MHC class II molecule. No strict limitation is imposed on the number of amino acid residues in the class II peptide, so long as a portion essential to class II peptide is included. The class II peptide is fitted in the peptide-binding groove of the MHC class II molecule, and protruded portions are generally cut by a peptidase. MHC class II molecules are present in antigen-presenting cells involved in immunoresponse, such as dendritic cells, B cells, macrophages, and thymic epithelial cells. A peptide-MHC class II complex formed in dendritic cells can activate naive CD4⁺ T cells. When the activated CD4⁺ T cells recognize a class II peptide presented by the peptide-MHC class II complex formed on the surfaces of B cells, the CD4⁺ T cells (Th2 cells) secrete a cytokine, whereby the isotype of the antibody to be produced by the B cells is determined. As a result, humoral immunity is induced. When the activated CD4⁺ T cells (Th1 cells) recognize a class II peptide presented by the peptide-MHC class II complex formed in macrophages, the CD4⁺ T cells activate macrophages, and a part of the cells secrete a cytokine, whereby the target pathogenic microorganism present in the endoplasmic reticulums of the macrophages is broken. Thus, cell-mediated immunity is induced by MHC class II in combination with a class II peptide. Examples of the cytokine involved in the action of Th1 cells include IFN-γ, IL-12, and TNF-α. Examples of the cytokine involved in the action of Th2 cells include IFN-γ, IL-12, IL-4, IL-5, IL-6, IL-10, and IL-12.

As shown in the below-mentioned Examples, when immunization is performed by use of class II peptide, not only the effect due to the function of humoral immunity (i.e., rise in IgG titer and IgA titer) and but also the effect due to the function of cell-mediated immunity (i.e., selective removal of virus-infected cells) is observed, and, by virtue of their synergistic effect, a dramatic reduction of the number of virus particles in the tissue is observed. This phenomenon shows that class II peptide intrinsically inducing humoral immunity is also involved in induction of cell-mediated immunity through an intercellular network based on secretion of physiologically active substances.

As also described above, differing from the class I peptide, the class II peptide has no amino acid residue which plays an important role in binding to an MHC class II molecule at each terminal of the peptide. Thus, terminals of the class II peptide cannot bind to the MHC class II molecule. Instead, the class II peptide lies in an extended conformation along the peptide-binding groove of the MHC class II molecule. It is held there both by peptide side chains that protrude into shallow and deep pockets lined by polymorphic amino acid residues, and by interactions between the peptide backbone and the side chains of conserved amino acids that line the peptide-binding groove in all MHC class II molecules. Analysis data show that amino acid side chains at the positions defined as the first, the fourth, the sixth, and the ninth positions of the class II peptide can be held in the aforementioned pockets. Regarding the amino acid residues of the class II peptide, the following positional tendency in characteristics are found. Specifically, the amino acid residue at the first position is hydrophobic (i.e., tyrosine, leucine, phenylalanine, or isoleucine); the amino acid residue at the fourth position is negatively charged (i.e., aspartic acid or glutamic acid); the amino acid residue at the sixth position is basic (i.e., lysine, asparagine, histidine, or glutamine); and the amino acid residue at the ninth position is hydrophobic (i.e., tyrosine, leucine, phenylalanine, or isoleucine). However, an exception for the tendency is also observed. Thus, the pockets of the MHC class II molecule accommodates a variety of amino acids, making it difficult to define anchor residues of the class II peptide and to predict which peptide will be able to bind a particular MHC class II molecule. However, by comparing the amino acid sequences of known binding class II peptides, it is possible to detect patterns of amino acids that permit binding to different allelic MHC class II variants, and to estimate the sequence motif of any amino acid residue of the peptide which binds to pockets of a peptide-binding groove of the MHC class II molecule.

In selection of the class II peptide, a candidate peptide sequence is selected from the nucleotide sequences of a gene encoding a target pathogenic microorganism or the amino acid sequences encoded by it by use of a computer program including an algorithm for selecting characteristics of the class II peptide, or a related database (e.g., http://tools.iedb.org/main/; http://tools.immuneepitope.org/main/datasets/; http://www.cbs.dtu.dk/services/NetMHCIIpan/logos.php; or Genetyx software, T cell epitope prediction) (all being commercially available), and the corresponding peptide is synthesized. The thus-synthesized peptide is tested in terms of activation of humoral immunity as an index, whereby a class II peptide of interest is obtained. Examples of the test method include ELISA (indirect assay). In one specific assay procedure, candidates of the class II peptide are fixed in wells of a multi-well plate, and a serum (a polyclonal antibody) from a mammal such as a human cured of the target disease is brought into contact with the wells. Further, a labeled secondary antibody to the polyclonal antibody is brought into contact with the wells. Through analyzing the label, whether or not a candidate of the class II peptide is a desired class II peptide can be determined.

Alternatively, as described above, selection of the class II peptide can be achieved by detecting a cytokine which is characteristic of function of MHC class II. The detection may be performed by use of a molecular needle carrying the test peptide as shown in the Examples. Details of the detection will be described below.

### (3) Linkage Peptide Formed of Class I Peptide and Class II Peptide

Class I peptide units and/or class II peptide units are linked to form a peptide (hereinafter may be referred to as a “linkage peptide”), which may be employed as a peptide (W) carried by a molecular needle. By tuning the type and number of peptide units in the linkage peptide to be carried, the mode and intensity of the immunity induced by a vaccine containing the molecular needle carrying the linkage peptide as an active ingredient can be modulated. Specifically, intensity of cell-mediated immunity can be elevated by increasing the number of class I peptide units, whereas intensity of humoral immunity can be elevated by increasing the number of class II peptide units.

A vaccine containing, as an active ingredient, a molecular needle carrying a linkage peptide formed only of class I peptide units is one embodiment of the vaccine 1 of the present invention. The class I peptide units forming the linkage peptide may be identical to or different from one another. Individual class I peptide units are preferably linked via a linker peptide. No particular limitation is imposed on the number of the amino acid residues forming the linker, but 3 to 10 residues are generally preferred. Also, no particular limitation is imposed on the number of class I peptide units forming the linkage peptide, but 2 to 15 units are generally preferred, with 2 to 5 units being more preferred.

A vaccine containing, as an active ingredient, a molecular needle carrying a linkage peptide formed only of class II peptide units is one embodiment of the vaccine 2 of the present invention. The class II peptide units forming the linkage peptide may be identical to or different from one another. Individual class II peptide units are preferably linked via a linker peptide. No particular limitation is imposed on the number of the amino acid residues forming the linker, but 3 to 10 residues are generally preferred. Also, no particular limitation is imposed on the number of class II peptide units forming the linkage peptide, but 2 to 15 units are generally preferred, with 2 to 5 units being more preferred.

A vaccine containing, as an active ingredient, a molecular needle carrying a linkage peptide formed of both class I peptide unit (s) and class II peptide unit (s) is one embodiment of the vaccine 3 of the present invention. The class I peptide units and the class II peptide units forming the linkage peptide may be identical to or different from one another, respectively. Individual class I peptide unit (s) and class II peptide unit (s) are preferably linked via a linker peptide. No particular limitation is imposed on the number of the amino acid residues forming the linker, but 3 to 10 residues are generally preferred. Also, no particular limitation is imposed on the number of class I peptide units and class II peptide units forming the linkage peptide, but 2 to 15 units are generally preferred, with 2 to 5 units being more preferred.

### (4) Peptide Functioning as Both Class I Peptide and Class II Peptide

In the living body, cell-mediated immunity function and humoral immunity function are in good balance to cope with infection with a microorganism. Particularly when the number of amino acids forming the peptide is in excess of 8 which may be a class II peptide, the class I peptide and class II peptide may also be present as motifs overlapping each other in the peptide or as motifs in separate regions of the peptide. When the peptide exerts a function as the class I peptide, the unnecessary portion other than the essential region of the class I peptide is thought to be digested by an exopeptidase in the endoplasmic reticulums, as described above.

The peptide functioning both the class I peptide and class II peptide has at least 8 amino acid residues which is the lower limit for the class II peptide.

### [7] The Information Acquisition Method of the Present Invention

As described above, it is a considerably important theme for the development of a new vaccine that a peptide or a protein which originates from a pathogenic microorganism and which less causes cytokine storm or the like is identified in determination of the immunogen of the vaccine. Meanwhile, the importance of acquiring information about whether the immunogen is classified into MHC class I or MHC class II is described above.

The present inventors have found that the object; i.e., prevention of the cytokine storm and determination of MHC class, can be solved by immunizing an animal with a molecular needle carrying a test peptide or protein and monitoring the change in secretion of a physiologically active substance such as a cytokine, after the immunization. The invention has been accomplished on the basis of this finding.

Accordingly, the present invention includes the following method for acquiring information (hereinafter referred to as “the information acquisition method of the present invention”).

The information acquisition method of the present invention includes:
preparing [A] a trimer or hexamer of a composite protein represented by the following amino acid sequence of formula (1-2):

$${W}_{2} - {L}_{1} - {X}_{n} - Y \qquad ( 1 - 2 )$$

[wherein W₂ represents an amino acid sequence of a peptide or protein which originates from a pathogenic microorganism serving as a test immunogen; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):

$${Y}_{1} - {L}_{2} - {Y}_{2} - {Y}_{3} \qquad ( 2 )$$

[wherein Y₁ represents any one amino acid sequence selected from the group consisting of SEQ ID NOS: 2 to 5; Y₂ represents any one amino acid sequence selected from the group consisting of SEQ ID NOS: 6 to 9; L₂ represents a second linker sequence having 0 to 30 amino acids; Y₃ represents an amino acid sequence for modification; and either of Y₂ and Y₃ may be absent],
wherein the amino acid sequence represented by Xₙ, Y₁, or Y₂ may include a modified amino acid sequence thereof obtained by deleting, substituting, or adding one or more amino acid residues from, in, or to the original amino acid sequence;
immunizing a test animal with the trimer or hexamer [A]; isolating immunocompetent cells of the thus-immunized test animal from the body of the test animal; quantitating one or more physiologically active substances present in the isolated immunocompetent cells; subsequently, infecting the isolated immunocompetent cells with a target pathogenic microorganism; quantitating the physiologically active substances present in the infected immunocompetent cells; and acquiring information about secretion of the physiologically active substances after immunization with the test immunogen W₂, on the basis of, as an index, a change in the level of each physiologically active substance before and after infection, obtained from the two quantitation values.

In other words, the information acquisition method of the present invention may also be expressed as “a detection method which includes immunizing a test animal with the trimer or hexamer [A]; isolating immunocompetent cells of the thus-immunized test animal from the body of the test animal; quantitating one or more physiologically active substances present in the isolated immunocompetent cells; subsequently, infecting the isolated immunocompetent cells with a target pathogenic microorganism; quantitating the physiologically active substances present in the infected immunocompetent cells; and detecting the secretion state of the physiologically active substances after immunization with the test immunogen W₂, on the basis of, as an index, a change in the level of each physiologically active substance before and after infection, obtained from the two quantitation values.”

The definition of “L₁, X, Y, n, Y₁, L₂, Y₂, and Y₃” in formula (1-2) is the same as in formula (1-1) above, and W₂ is “an amino acid sequence of a peptide or protein which originates from a pathogenic microorganism as a test immunogen.”

Immunocompetent cells are collected from a biological tissue including imunocompetent cells (e.g., the spleen, marrow, thymus, and lymph nodes), or from body fluid such as blood or lymph. No particular limitation is imposed on the test animal which is a target of the information acquisition method of the present invention. Examples of preferred test animals include mammals such as rats, mice, hamsters, monkeys, dogs, and cats. The animals may be humans. However, in this case, immunocompetent cells must be collected from body fluid (e.g., blood, lymph, or spinal fluid) sampled removed from the living body, or collected from a biological tissue removed by biopsy or from a living body upon surgery for the purpose of survival or lifesaving.

Examples of such immunocompetent cells include, but are not limited to, T cells, B cells, macrophages, mastocytes, eosinophils, neutrophils, basophils, dendritic cells, and a mixture thereof.

No particular limitation is imposed on the physiologically active substance which is a target of information acquisition, so long as the substance is detectable. The target substance preferably includes a cytokine or chemokine, which is secreted in the course of working of the immune system of the living body.

As described in the Examples below, in one preferred embodiment, the information about secretion of the physiologically active substance is information about whether or not a test immunogen W₂ is classified into MHC class I or MHC class II.

As described above, examples of the signal showing that the test immunogen is classified into MHC class I include activation signals of IFN-γ, IL-2, IL-12, MIP-1a, MIP-1b, TNF-α, IL-10, perforin, granzyme A, granzyme B, RANTES, etc. Examples of the signal showing that the test immunogen is classified into MHC class II include activation signals of IFN-γ, IL-12, TNF-α, etc. which are cytokines involving the function of Th1 cells, and activation signals of IFN-γ, IL-12, IL-4, IL-5, IL-6, IL-10, IL-12, etc. which are cytokines involving the function of Th2 cells. Such an activation signal is generally equivalent to the change in secretion level of a target physiologically active substance attributed to infection with a target pathogenic microorganism, and the change is generally an increase. No particular limitation is imposed on the threshold value for evaluating the increase as a positive signal. In one case, as shown in the Examples, when a ratio of secretion level after infection to that before infection greater than 2 is employed as an activation signal, the ratio may serve as a suitable threshold value which can appropriately (i.e., correctly without noise) show activation of secretion of a physiologically active substance. However, the threshold value may be freely predetermined in accordance with the purpose of information acquisition, and the type and number of the physiologically active substances to be detected. Particularly when a variety of or a large number of physiologically active substances are detected, for example, five or more types of physiologically active substances are detected, determination as MHC class I or II is not always based on the ratio greater than the threshold value for all types (100%) of the targeted physiologically active substances. Alternatively, MHC class determination can be accomplished based on the ratio greater than the threshold value for about 80% (e.g., 4 types in 5 types) of the targeted physiologically active substances.

Similar to other embodiments of the present invention, examples of the pathogenic microorganism which is a target of the information acquisition method of the present invention include a virus, a bacterium, a fungus, and a protozoa. Among them, a virus is preferred.

The information obtained through the information acquisition method of the present invention relates to a physiologically active substance secretion characteristic upon infection with a target microorganism (a test immunogen) and is of great value. The information is important and useful for selecting the immunogen of a vaccine, for example, selecting a more effective immunogen from the viewpoints of suppression of cytokine storm and choice of MHC class I or II.

Determination of MHC class I or II is valuable for examining validity of choice of the immunogen in advance by analysis with computer software or by known related information. The information acquisition method of the present invention is useful not only in the case where the active ingredient of a vaccine in development is a peptide or a protein, but also in the case where it is a nucleic acid.

### Effects of the Invention

The present invention can provide the following (1) to (4). (1) A composite protein (a monomer) formed of a molecular needle carrying, as an immunogen, a peptide compatible with MHC class I and/or MHC class II. The immunogen is “one or more species of a peptide including one or more peptides which are compatible with MHC class I and which originate from a pathogenic microorganism,” “one or more species of a peptide including one or more peptides which are compatible with MHC class II and which originate from a pathogenic microorganism,” or “one or more species of a peptide including one or more peptides which are compatible with MHC class I and which originate from a pathogenic microorganism and one or more peptides which are compatible with MHC class II and which originate from the pathogenic microorganism.” The composite protein can be used as an essential unit of an active ingredient (i.e., an infection-protective antigen) of a component vaccine that can efficiently introduce the immunogen into target tissue cells through transmucosal, percutaneous, subcutaneous, intradermal, or intramuscular administration. (2) An associated product of the composite protein which can be used as the active ingredient (i.e., an infection-protective antigen). (3) An MHC class I-activating component vaccine which contains the associated product as an active ingredient and which selectively induces cell-mediated immunity (the vaccine 1 of the present invention); an MHC class II-activating component vaccine which preferentially induces humoral immunity (the vaccine 2 of the present invention); or an MHC class I and II-activating component vaccine which contains the associated product as an active ingredient and which induces both cell-mediated immunity and humoral immunity (the vaccine 3 of the present invention), and a vector for gene expression and a transformant in relation to the vaccines. (4) A method for acquiring information about secretion of a physiologically active substance. The information acquisition method is remarkably useful in development of a vaccine against a target pathogenic microorganism, particularly in determination of the MHC class of the immunogen, and evaluation of a physiologically active substance (e.g., cytokine) secretion characteristic upon infection with the pathogenic microorganism (immunogen).

### BRIEF DESCRIPTION OF THE DRAWINGS

FIG. 1 A scheme of forming the associated products of the present invention (trimer and hexamer) from the composite protein of the present invention.

FIG. 2 MALDI-TOF mass spectra of PN-sRL-VPg: (a) a signal of monomer, (b) a signal of trimer, and (c) a signal of hexamer (i.e., trimer-dimer). A rough sketch showing the association state of the composite protein of the present invention (i.e., monomer) is attached to each spectrum chart.

FIG. 3 MALDI-TOF mass spectra of PN-sFL-VPg: (a) a signal of monomer, (b) a signal of trimer, and (c) a signal of hexamer (i.e., trimer-dimer). A rough sketch showing the association state of the composite protein of the present invention (i.e., monomer) is attached to each spectrum chart.

FIG. 4 Schematic structure of an RS virus gene.

FIG. 5 A schematic view of a plasmid structure “pET29b (+)/Lpep-PN” produced by changing Vpg to a gene fragment encoding the test peptide of Example 1 through In-Fusion cloning.

FIG. 6 Graphs showing an IgA induction effect by nasal inoculation of a component vaccine employed in Example 1 (3 weeks after inoculation). The active ingredient of the vaccine is the composite protein associated product of the present invention carrying a test peptide in L protein (i.e., a non-structural protein of RS virus).

FIG. 7 Graphs showing an IgG induction effect by nasal inoculation of a component vaccine employed in Example 1 (3 weeks after inoculation). The active ingredient of the vaccine is the composite protein associated product of the present invention carrying a test peptide in L protein (i.e., a non-structural protein of RS virus).

FIG. 8 A graph showing an effect of reducing the number of the infected RS virus in the lungs through nasal inoculation of a component vaccine employed in Example 1. The active ingredient of the vaccine is the composite protein associated product of the present invention carrying a test peptide in L protein (i.e., a non-structural protein of RS virus).

FIG. 9 A graph showing the change over time in body weight of test hamsters after infection with a novel coronavirus in Example 2.

FIG. 10 A graph showing the amount of virus in the lung tissue of a test hamster after infection with a novel coronavirus in Example 2.

### MODES FOR CARRYING OUT THE INVENTION

### (1) The Composite Protein of the Present Invention

The composite protein of the present invention is represented by the amino acid sequence of the following formula (1):

$$W - {L}_{1} - {X}_{n} - Y \qquad ( 1 )$$

[wherein W represents one or more amino acid sequences of a peptide including one or more peptides selected from peptides which are compatible with MHC class I and which originate from a pathogenic microorganism and/or peptides which are compatible with MHC class II and which originate from the pathogenic microorganism, as an immunogen; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 3].

The segment W which is an immunogen, is one or more amino acid sequences of a peptide including one or more peptides selected from peptides which are compatible with MHC class I and which originate from a pathogenic microorganism and/or peptides which are compatible with MHC class II and which originate from the pathogenic microorganism. Regarding the immunogen, the concept “a peptide including two or more peptides (a plurality of peptides)” implied in “a peptide including one or more peptides” refers to, for example, the case in which a plurality of class I peptides and/or class II peptides are included as a linked form or the like in one peptide. The expression “including” may also encompass the case of including, in addition to the class I peptide or class II peptide, an intentionally inserted modification amino acid sequence, and the case of linking a linker amino acid sequence to form W.

It is possible to link only class I peptide units or only class II peptide units, or to link class I peptide unit (s) and class II peptide unit (s). The linker peptide used for linking the units preferably has 3 to 10 amino acid residues, more preferably 4 to 6 amino acid residues. The linked peptide is suitably formed of 2 to 15 class I peptide units and/or class II peptide units. Specific examples of the linker peptide include, but are not limited to, “GGGG” (SEQ ID NO: 58), “GGGGS” (SEQ ID NO: 15), PAPAP (SEQ ID NO: 16), and “SNSSSVPGG” (SEQ ID NO: 14) (each amino acid being represented by a single letter abbreviation).

The aforementioned W may be formed by linking identical class I peptide units or class II peptide units (W1) by the mediation of linker peptides (e.g., “W1+GGGGS+W1+GGGGS+W1”). Alternatively, the aforementioned W may be formed by linking different class I peptide unit (s) and/or class II peptide unit (s) by the mediation of linker peptides (e.g., “W1+GGGGS+W2+GGGGS+W3”).

The first linker sequence L₁ is required for appropriately maintaining the distance between the immunogen W and the cell introduction domain Y, to thereby suppress steric hindrance. As described above, the number of amino acid residues in linker L₁ is 0 to 100, preferably 4 to 40. No particular limitation is imposed on the specific amino acid sequence, and examples thereof include (GGGGS)ₘ, (PAPAP)ₘ, and (SNSSSVPGG)ₘ [m represents the number of repetitions and is preferably an integer of 1 to 10, particularly preferably 1 to 3]. Needless to say, the above examples are non-limitative ones.

X is an amino acid residue represented by SEQ ID NO: 1, and the amino acid sequence Xₙ is a sequence of repeating unit Xs with the number of repetitions n (n is an integer). The mode of repetition is linear repetition. The case of X₂ means “X-X” (wherein symbol “-” denotes a peptide bond). Also, in the repeated sequence Xₙ, the amino acid sequence may be modified in the aforementioned manner. As mentioned above, the number n is an integer of 1 to 3. The number n is preferably 1 but may be 2 or 3. The repeated sequence Xₙ (n=2 or 3) is employed primarily for consistently maintaining the suitable distance between the cell introduction domain Y and the immunogen W depending on the dimension and characteristics of the immunogen W.

The cell introduction domain Y is a basic structure of the molecular needle and corresponds to the tail needle (pin) of a bacteriophage (i.e., cell-penetrating part). The domain Y is a protein represented by the following formula (2):

$${Y}_{1} - {L}_{2} - {Y}_{2} - {Y}_{3} \qquad ( 2 )$$

[wherein Y₁ represents an amino acid sequence selected from the group consisting of SEQ ID NOS: 2 to 5; Y₂ represents an amino acid sequence selected from the group consisting of SEQ ID NOS: 6 to 9; L₂ represents a second linker sequence having 0 to 30 amino acids; Y₃ represents an amino acid sequence for modification of interest; and Y₂ or Y₃ may be absent].

In Y₁ of formula (2), the amino acid sequence from the end of the N-terminal side to the 32 amino acid residue (32 Leu) corresponds to the amino acid sequences of a triple helix β-sheet structure of the bacteriophage T4. The N-terminal amino acid of valine (1 Val) may also be leucine (1 Leu). The remaining C-terminal amino acid sequence is an amino acid sequence of the needle protein of the bacteriophage on the C-terminal side. Examples of the amino acid sequence which may be used on the C-terminal side of Y₁ include an amino acid sequence of gp5 of bacteriophage T4, an amino acid sequence of gpV of bacteriophage P2, an amino acid sequence of gp45 of bacteriophage Mu, and an amino acid sequence of gp138 of bacteriophage ϕ92. More specific examples of the amino acid sequence of Y₁ include the amino acid sequence (SEQ ID NO: 2) which has the amino acid sequence of gp5 of bacteriophage T4 on the C-terminal side, the amino acid sequence (SEQ ID NO: 3) which has the amino acid sequence of gpV of bacteriophage P2 on the C-terminal side, the amino acid sequence (SEQ ID NO: 4) which has the amino acid sequence of gp45 of bacteriophage Mu on the C-terminal side, and the amino acid sequence (SEQ ID NO: 5) which has the amino acid sequence of gp138 of bacteriophage ϕ92 on the C-terminal side. The nucleotide sequence encoding the amino acid sequence of Y₁ may be selected in accordance with a generally known amino acid-nucleobase relationship.

Y₂ in formula (2) is an amino acid sequence of a domain “foldon” of bacteriophage T4 or an amino acid sequence of a domain “tip” of bacteriophage P2, bacteriophage Mu, or bacteriophage ϕ92. The foldon or tip is a domain forming the tip portion of the molecular needle structure (i.e., fibritin) of bacteriophage. In formula (2), Y₂ is not necessarily present. However, when the amino acid sequence of the foldon or the tip is included, the efficiency of incorporation of the molecular needle to the cell membrane can be enhanced. Thus, the presence of Y₂ is remarkably preferred. The amino acid sequence of the foldon of bacteriophage T4 is represented by SEQ ID NO: 6. The nucleotide sequence encoding the amino acid sequence may be selected in accordance with a generally known amino acid-nucleobase relationship.

The amino acid sequence of the tip of bacteriophage P2 is represented by SEQ ID NO: 7. The nucleotide sequence encoding the amino acid sequence may be selected in accordance with a generally known amino acid-nucleobase relationship. The amino acid sequence of the tip of bacteriophage Mu is represented by SEQ ID NO: 8. The nucleotide sequence encoding the amino acid sequence may be selected in accordance with a generally known amino acid-nucleobase relationship. The amino acid sequence of the tip of bacteriophage ϕ92 is represented by SEQ ID NO: 9. The nucleotide sequence encoding the amino acid sequence may be selected in accordance with a generally known amino acid-nucleobase relationship.

L₂ serves as a second linker intervening between Y₁ and Y₂. The number of amino acid residues in linker L₂ is 0 to 30, preferably 0 to 5. The number of amino acid residues in linker L₂ of 0 means the absence of second linker L₂.

Y₃ is an amino acid sequence for modification and may be selectively incorporated into Y. The modification amino acid sequence is added in order to purify or protect protein or for another reason, and examples thereof include tag peptides such as a histidine tag, a GST tag, and a FLAG tag. The modification amino acid sequence Y₃ preferably includes a histidine tag, both in a protein purification step and from the kinetic viewpoint of introducing a trimer or hexamer of the composite protein as a vaccine active ingredient into target cells. Also, an appropriate linker sequence may be incorporated into Y₃. Such an additional linker sequence itself may also be a component of the amino acid sequence of Y₃.

The composite protein of the present invention may be produced through a known method; specifically, a genetic engineering process or chemical synthesis. The entirety of the composite protein of the present invention may be produced in a single process. Alternatively, segments of the composite protein are individually produced, and the produced segments are linked through a chemical modification method to produce the composite protein. In linking polypeptides by the mediation of a linker (e.g., L₁ or L₂), a lysine residue or a cysteine residue of one polypeptide can be linked to that of another polypeptide by the mediation of a linker having a succinimido group or a maleimido group.

In a genetic engineering process, a nucleic acid fragment encoding the entirety or a part of the target composite protein of the present invention may be introduced into host cells (e.g., Escherichia coli, yeast, insect cells, and animal cells) and expressed, or may be expressed in a cell-free expression system such as an E. coli extract, a rabbit reticulocyte extract, or a wheat germ extract. Any nucleic acid expression vector suited for the expression system may be used. Examples of the expression vector include pET (for expression in E. coli), pAUR (for expression in yeast), pIEx-1 (for expression in insect cells), pBApo-CMV (for expression in animal cells), and pF3A (for expression in wheat germ extract).

Regarding chemical synthesis, any known method for chemically synthesizing peptides may be employed. More specifically, the entirety or a part of the composite protein of the present invention may be produced through any established customary method (e.g., liquid-phase peptide synthesis or solid-phase peptide synthesis). As a solid-phase peptide synthesis method generally recognized as a preferred chemical synthesis method, a Boc solid method or an Fmoc solid method may be employed. Also, as described above, a ligation technique may be employed in accordance with need. Needless to say, each amino acid may be produced through a known method, and a commercial product thereof may be used.

### (2) The Associated Product of the Present Invention

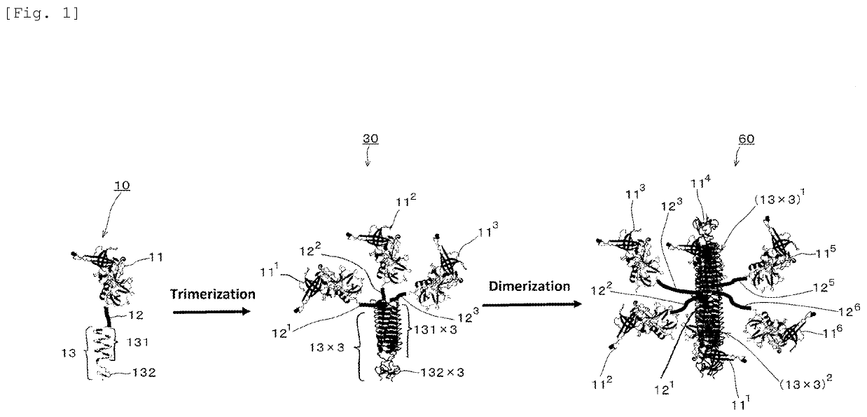

FIG. 1 is a scheme of forming the associated products of the present invention (trimer and hexamer) from the composite protein of the present invention. In FIG. 1, reference numeral 10 denotes the composite protein of the present invention in the form of monomer. Reference numeral 20 denotes the trimer of the present invention, and reference numeral 30 denotes the hexamer of the present invention.

The composite protein 10 of the present invention is formed of a “molecular needle domain 13 corresponding to Y in formula (1)” in which a “base part 131 corresponding to Xₙ and Y₁ in formula (2)” is linked to a “foldon 132 corresponding to Y₂ in formula (2),” and an “immunogen 11 corresponding to W in formula (1),” wherein the molecular needle domain 13 and the immunogen 11 are linked together via a “linker 12 corresponding to L₁ in formula (1). Notably, a linker other than linker 12, and the modification sequence corresponding to Y₃ in formula (2) are not illustrated. The composite protein 10 of the present invention per se substantially exhibits no function of penetrating the cell membrane of the target tissue cells.

The trimer 30 is an associated product of three molecules of the aforementioned composite protein 10 serving as a monomer via a spontaneous association process. In the trimer 30, 3 units of the molecular needle domain 13 are combined with association via formation of C-terminal-C-terminal bonds, to thereby provide a trimer parallel β-sheet structure, and a helix structure of the β-sheet structure per se (i.e., triple helix β-sheet structure), which corresponds to a needle structure. As a result, a molecular needle 13×3 is formed. The molecular needle 13×3 is composed of a basic structure 131×3 and a foldon aggregate 132×3. In this way, a “molecular needle” having a target tissue cell membrane penetration function is formed through trimerization and a self-assembly process. Three linkers (12¹, 12², 12³) originating from the respective monomers, and three immunogen portions (11¹, 11², 11³) linked to the respective linkers are present in the outside area of the molecular needle 13×3.

The hexamer 60 is formed through linking 2 units of the trimer 30, with formation of a bond between the N-terminals of the molecular needle basic parts (13×3)¹ and (13×3)². The hexamer 60 also exhibits a target tissue cell membrane penetration function. Six linkers (12¹, 12², 12³, 12⁵, 12⁶, 12⁴ (not illustrated)) originating from the respective trimers, and six immunogen portions (11¹, 11², 11³, 11⁵, 11⁶, 11⁴ (not illustrated) linked to the respective linkers are present in the outside area of two molecular needles (13×3)¹ and (13×3)².

Trimerization of the composite protein 10 of the present invention to form the trimer 30, and further dimerization of the trimer 30 to form the hexamer 60 spontaneously proceed in aqueous liquid. The formed trimer or hexamer exists in a stable state. The stability of the trimer or hexamer is remarkably high. For example, the trimer or hexamer is stable in an aqueous liquid at 100° C., in an aqueous liquid at a pH of 2 to 11, or in an aqueous liquid containing 50 to 70 vol. % of organic solvent. In addition, the trimer or hexamer is excellent in safety. When being isolated from the aqueous liquid and dried, the trimer or hexamer is highly stable and exhibits an excellent cell membrane penetration function.

As described above, transformation of the composite protein of the present invention to the associated product of interest proceeds spontaneously. Generally, most becomes the hexamer (final product), but some remains as the trimer.

### (3) The Vaccine of the Present Invention

The vaccine of the present invention contains, as an active ingredient, the associated product of the present invention which exhibits an excellent cell penetration function and immunogenicity. Thus, when administered to target tissue cells via subcutaneous administration, intradermal administration, percutaneous administration, mucosal administration, or intramuscular administration, the immunogen can be efficiently transferred to the target tissue cells, wherein the immunogen is one or more species of a peptide including one or more peptides which are compatible with MHC class I and which originate from a pathogenic microorganism (the vaccine 1 of the present invention); one or more species of a peptide including one or more peptides which are compatible with MHC class II and which originate from a pathogenic microorganism (the vaccine 2 of the present invention); or one or more species of a peptide including one or more peptides which are compatible with MHC class I and which originate from a pathogenic microorganism and one or more peptides which are compatible with MHC class II and which originate from the pathogenic microorganism (the vaccine 3 of the present invention). The vaccine 1 of the present invention can selectively induce cell-mediated immunity. The vaccine 2 of the present invention can preferentially induce humoral immunity. The vaccine 3 of the present invention can induce cell-mediated immunity and humoral immunity, while the balance between two types of immunity is appropriately modulated. In addition, the efficacy and safety of the component vaccine against a pathogenic microorganism (e.g., a virus or a bacterium) via subcutaneous administration, intradermal administration, percutaneous administration, mucosal administration, or intramuscular administration can be enhanced, which leads to the characteristic feature of the component vaccine of the invention that the vaccine may be used as an adjuvant-free vaccine. No particular limitation is imposed on the target mucosal tissue when the vaccine is mucosally administered, and the target mucosal tissue may be freely selected in accordance with the site affected by the target pathogenic microorganism (in particular, a virus) and other factors. Examples of the target mucosal tissue include the nasal mucous membrane, throat mucous membrane, oral mucous membrane, bronchial mucous membrane, alimentary canal mucous membrane, and vaginal mucous membrane. In the case of a virus causing respiratory tract inflammation (cold) (e.g., RS virus, a corona virus (including a β-corona virus such as a novel coronavirus), or an influenza virus), suitable targets include the nasal mucous membrane, throat mucous membrane, oral mucous membrane, bronchial mucous membrane, sublingual mucous membrane, and lung mucous membrane.

The vaccine of the present invention is provided as a pharmaceutical composition for subcutaneous administration, intradermal administration, percutaneous administration, mucosal administration, or intramuscular administration, the composition containing the aforementioned associated product of the present invention as an active ingredient (protective antigen). The associated product of the present invention may be mixed with and suspended in a buffer or the like upon use, and the resulting liquid agent may be subcutaneously, intradermally, percutaneously, mucosally, or intramuscularly administered. Thus, the pharmaceutical composition also encompasses the form of the associated product itself. In the case of the aforementioned mucosal administration, suitable agent forms include spray (e.g., aerosol or spray), capsule, and coating.

The vaccine of the present invention may contain a plurality of different associated products of the present invention as active ingredients.

The vaccine of the present invention may be prepared into a pharmaceutical composition by blending the associated product of the present invention serving as an essential active ingredient (infection protective antigen) with an optional molecular needle to which another component peptide of the target pathogenic microorganism has been attached, and a further optional adjuvant and an appropriate pharmaceutical carrier. Needless to say, the vaccine of the present invention may be prepared into an adjuvant-free formulation. The pharmaceutical carrier may be selected in accordance with the form of use. Examples of the pharmaceutical carrier include a filler, an extender, a binder, a humectant, a disintegrant, a surfactant, an excipient, and diluent. The form of the composition is generally liquid, but may be a dry product, powder, granule, and the like, which are diluted with liquid upon use.

In the vaccine of the present invention, the amount of the associated product of the present invention (i.e., the total amount of the associated products employed, including different associated products or an associated product of a molecular needle carrying a component protein or peptide of another pathogenic microorganism) is not necessarily fixed and may be appropriately tuned. Generally, the associated product of the present invention is suitably used as a liquid formulation containing the product in an amount of 1 to 10 mass % upon administration. The appropriate single dose of administration (inoculation) is about 0.01 μg to about 10 mg for an adult. As needed, the initial inoculation may be appropriately combined with a booster inoculation. The administration (inoculation) may be carried out one or more times.

When the plurality of associated products of the present invention are used in combination, the proportions among the associated products may be predetermined in accordance with the type of the target pathogenic microorganism or the purpose of use. For example, when both cell-mediated immunity and humoral immunity are to be induced to a target pathogenic microorganism which tends to cause disease enhancement, the associated product 1 of the present invention that can induce cell-mediated immunity is used as a primary active ingredient in an effective amount, and the associated product 2 of the present invention is used as a secondary active ingredient in a small amount. The specific ratio of the two associated products may be adjusted on a case-by-case basis.

### EXAMPLES

The present invention will next be described in detail by way of example.

The Examples are provided so as to demonstrate the advantageous feature of the associated product 2 of the present invention as the active ingredient of a component vaccine targeted to a specific virus. In view of difficulty in current production and usefulness of a vaccine, RS virus (RSV), also called orthopneumovirus, was taken as an example of the virus.

RS virus is widely identified in the world and can cause lifelong apparent infection of a human subject regardless of age. Particularly in infancy, RS virus is a serious pathogen. Although babies and infants have an antibody to RS virus, which antibody has been transferred from the mother's body, most severe symptoms may be induced in a period from several weeks to several months after birth. In the case of low birth-weight infants, and infants having an underlying cardiopulmonary disease or immunodeficiency, the symptoms tend to become more grave. Thus, RS virus has a significant impact on clinical settings and public health.

Currently, there is no authorized RSV vaccine. Previously, some clinical tests were conducted with a formalin-inactivated vaccine. However, the trials failed, since the symptoms of the vaccinated groups were more aggravated than those of the control group. One pharmaceutical product to cope with RSV infection is Palivizumab, which is a human monoclonal antibody prophylactically administered to a subject in need thereof. Thus, there is demand for an effective vaccine (cited from web page of the National Institute of Infectious Disease, Japan).

[Referential Example] Preparation of Vector Including a Nucleic Acid Fragment Encoding a Composite Protein which Serves as a Template of the Vector for Producing an Associated Product Employed in the Examples

### (a) Introduction of Preparing Template Vector

Through a genetic engineering technique, a vector including a nucleic acid fragment encoding a composite protein serving as an immunogen to be attached was prepared. The immunogen is a non-structural protein of human norovirus GII.4 (i.e., LM14-2 variant), which is VPg (viral protein genome-linked). VPg is a non-structural protein included in open reading frame 1 (ORF1) of the norovirus genome. ORF1 encodes a series of non-structural proteins of nororvirus including N-terminal protein, NTPase (p48), p22 (3A-like), VPg, protease, and RNA-dependent RNA polymerase (RdRp). After completion of total translation of ORF1, individual non-structural proteins are provided by the corresponding protease, which proteins serve as mature products. Among these mature products, VPg has been found to play an essential role in replication of the norovirus genome via translation from the genomic RNA and the subgenomic RNA. Actually, VPg functions as a cap substitute in mobilization of ribosomes. VPg of LM14-2 variant employed as an immunogen in this Example has an amino acid sequence represented by SEQ ID NO: 10 (Notably, Met on the N-terminal originates from start codon ATG). The nucleotide sequence encoding the amino acid sequence may be selected in accordance with a generally known amino acid-nucleobase relationship.

All the reagents employed in the above step were purchased from commercial suppliers and used without additional purification. As a gene fragment of HNV-VPg, there was used a gene fragment included in a cDNA fragment (7, 639 bases: SEQ ID NO: 12) of human norovirus LM14-2 variant incorporated into plasmid pHuNOV-LM14-2F (1 to 12, 774 bases: SEQ ID NO: 11) provided by Katayama, Viral Infection Research Institute, Kitasato University. VPg is represented by a sequence formed of 399 bases (SEQ ID NO: 13) corresponding to the 2, 630th base to the 3,028th base of a CDNA fragment (7,639 bases) of the LM14-2 variant. Start codon ATG was attached to the 5′-terminal of the above sequence, and the product was used in gene expression.

UV-vis spectra were measured by means of SHIMADZU UV-2400PC UV-vis spectrometer. MALDI-TOF mass spectra were measured by means of Bruker ultraflextrme. In the MALDI-TOF-MS measurement, each sample was mixed with an equivolume of 70% (v/v) acetonitrile/water containing 0.03% (w/v) sinapic acid and 0.1% (v/v) trifluoroacetic acid. Gel permeation chromatography (GPC) was conducted by means of an HPLC system with a column (Asahipack GF-510HQ, Shodex, Tokyo, Japan).

### (b) Production of Plasmid for Forming “PN-VPg” Via Gene Expression

(b)-1: General

“PN-VPg” is a composite peptide represented by the aforementioned formula (3):

$${W}_{1} - {L}_{1} - {X}_{n} - Y , \qquad ( 3 )$$

wherein Y represents a cell introduction domain represented by formula (4):

$${Y}_{1} - {L}_{2} - {Y}_{2} - {Y}_{3} , \qquad ( 4 )$$

wherein L₁, Xₙ, Y, Y₁, L₂, Y₂, and Y₃ are the same as defined in formulae (1) and (2), and W₁ is an immunogen.

In the above formulas, the immunogen W₁ is “LM14-2 variant-VPg” represented by amino acid sequence SEQ ID NO: 10; the first linker L₁ is amino acid sequence SEQ ID NO: 14 (SNSSSVPGG), 15 (GGGGS), or 16 (PAPAP); the repeating unit of the repeating sequence Xₙ is amino acid sequence SEQ ID NO: 1; the number n is 1; the amino acid sequence of the molecular needle base Y₁ is amino acid sequence SEQ ID NO: 2; the second linker L₂ is “SVE”; the amino acid sequence of the foldon Y₂ is amino acid sequence SEQ ID NO: 6; and the amino acid sequence of the modification sequence Y₃ is amino acid sequence SEQ ID NO: 17 (VEHHHHHH).

The plasmid for forming PN-VPg via gene expression was constructed from a flexible linker (FL: SNSSSVPGG (SEQ ID NO: 14)) as a template, and two shorter linkers; a flexible linker (sFL: GGGGS (SEQ ID NO: 15)) and a rigid linker (sRL: PAPAP (SEQ ID NO: 16)). Through gene expression, an associated product generated spontaneously. The presence of a protein trimer and a protein hexamer was confirmed by analyzing the contents of the associated product. Based on the above, an RS virus component vaccine (i.e., a present target) was produced and tested. As a result, it was demonstrated that the associated product 3 of the present invention employing a peptide that functions both as MHC class I peptide and MHC II class peptide of RS virus was remarkably useful for providing a component vaccine.

(b)-2: Construction of Template Plasmid by Use of Flexible Linker (FL: SNSSSVPGG (SEQ ID NO: 14))

A VPg segment obtained from LM14-2 plasmid was amplified through polymerase chain reaction (PCR) by use of a gene amplification primer VPg_F (with an NdeI restriction enzyme site: ACGCCATATGGGCAAGAAAGGGAAGAACAAGTCC (SEQ ID NO: 18)) and a gene amplification primer VPg_R (with an EcoRI restriction enzyme site: GCTCGAATTCGACTCAAAGTTGAGTTTCTCATTGTAGTCAACAC (SEQ ID NO: 19)). Thereafter, the PCR product was cloned into plasmid pKN1-1 (plasmid for GFP-gp5f expression) (Patent Document 2) digested by NdeI-EcoRI.

The plasmid pKN1-1 was yielded according to the disclosure of Patent Document 2. In the specific procedure, firstly, the gene fragment corresponding to the 461th to the 484th amino acid residues of the wac protein of T4 phage was amplified through PCR from the T4 phage genome, followed by cloning to pUC18, to thereby yield a gene encoding the foldon. Subsequently, the resultant plasmid was cut by restriction enzymes EcoRI and SalI, and the product was inserted into a plasmid pET29b (Novagen) treated by EcoRI and XhoI, to thereby yield plasmid pMTf1-3. Also, the gene fragment corresponding to the 474th to the 575th amino acid residues of gp5 of the T4 phage was amplified through PCR, followed by cloning to pUC18 from the T4 phage genome, to thereby yield a gene encoding gp5. Thereafter, the resultant plasmid was cut by restriction enzymes EcoRI and SalI, and the product was inserted into the aforementioned plasmid pMTf1-3 treated by EcoRI and XhoI, to thereby yield plasmid pKA176. Separately, the GFP expression vector provided by Takahasi of Gunma University was cut by restriction enzymes NdeI and EcoRI, to thereby yield a gene encoding the GFP. The product was inserted into the aforementioned plasmid pKA176 treated by NdeI and EcoRI.

The thus-cloned gene fragment was introduced into competent cells of E. coli BL21 (DE3), and the presence of the gene fragment was identified through DNA sequencing. Thus, formation of a plasmid structure including PN and VPg by the mediation of the flexible linker (SNSSSVPGG: SEQ ID NO: 15) “PN-FL-VPg” was confirmed.

(b)-3: Production of the Associated Products by Use of SFL/sRL Linker and Identification Thereof

Two primer combinations were individually used in inverted PCR employing the “PN-FL-VPg” as a template. One primer combination was a gene amplification primer VPgPA-F (with XhoI restriction site: CCGGCTCCGGCCCCACTCGAGGGAAGCAATACAATATTTGTACG (SEQ ID NO: 20)) and a gene amplification primer VPgPA-R (CTCAAAGTTGAGTTTCTCATTGTAGTCAACAC (SEQ ID NO: 21)), for incorporating a “shorter rigid linker (sRL: PAPAP)” represented by SEQ ID NO: 16 as linker L₁. The other primer combination was a gene amplification primer VPgGS-F (with an XhoI restriction site: GGAGGCGGGGGTTCACTCGAGGGAAGCAATACAATATTTGTACG (SEQ ID NO: 22)) and a gene amplification primer VPgGS-R (the same as that of the aforementioned VPgPA-R (SEQ ID NO: 21)), for incorporating a “shorter flexible linker (sFL: GGGGS)” represented by SEQ ID NO: 15 as linker L₁. As a result, a plasmid structure “PN-SRL-VPg” (wherein L₁ is a shorter rigid linker represented by SEQ ID NO: 16) and a plasmid structure “PN-sFL-VPg” (wherein L₁ is a shorter flexible linker represented by SEQ ID NO: 15) were constructed.

Subsequently, each of the two plasmids was introduced into DH5α competent cells. The thus-obtained vectors were identified through DNA nucleotide sequencing. Thereafter, PN-sRL-VPg and PN-sFL-VPg were formed via gene expression.

Each of the thus-formed “PN-SRL-VPg” and “PN-sFL-VPg” was applied to an Ni affinity column, and the protein contents were eluted with imidazole with a higher concentration gradient of 20 to 500 mM and 1 mM DTT. Fractions containing PN-VPg were combined, and the volume of the mixture was reduced to 5 mL. The product was dialyzed overnight against a 20 mM Tris-HCl buffer (pH: 8.0) at 4° C. by the mediation of a dialysis membrane. The thus-concentrated product was filtered through a 0.2-μm filter, and the filtered liquid was applied to a HiTrap Q column. Then, elution was performed with concentration-graded (0 to 1M) sodium chloride solution. Fractions containing PN-VPg (shorter linker) were combined, and the combined product was analyzed through Native PAGE and SDS-PAGE.

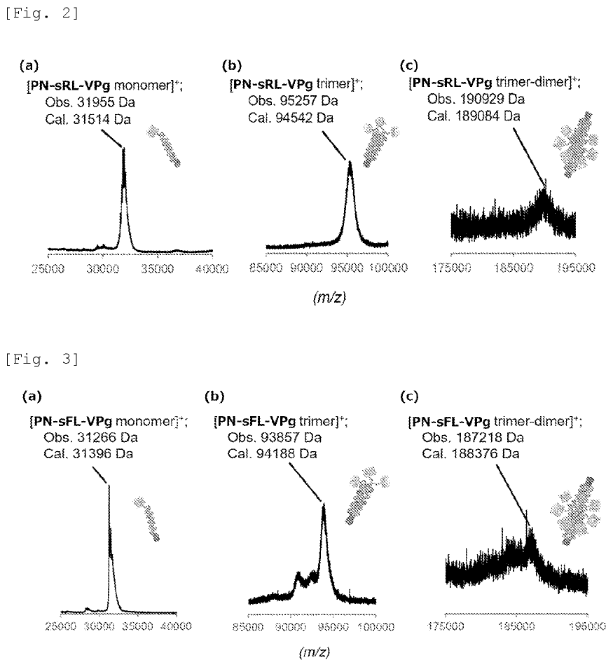

The SDS PAGE analysis showed a band attributable to a monomer having a calculated molecular weight of about 31 kDa, which corresponds to PN-VPg (sFL/SRL linker) (not illustrated). In addition, the MALDI-TOF mass spectra regarding PN-sRL-VPg showed a monomer signal (in FIG. 2 (a), m/z (observed): 31,955, m/z (calculated): 31,514), a trimer signal (in FIG. 2 (b), m/z (observed): 95,257, m/z (calculated): 94,542), and a hexamer (trimer-dimer) signal (in FIG. 2 (c), m/z (observed): 190,929, m/z (calculated): 189,084). Further, the MALDI-TOF mass spectra regarding PN-sFL-VPg showed a PN-sFL-VPg monomer signal (in FIG. 3 (a), m/z (observed): 31,266, m/z (calculated): 31,396), a trimer signal (in FIG. 3 (b), m/z (observed): 93,857, m/z (calculated): 94,188), and a hexamer (trimer-dimer) signal (in FIG. 3 (c), m/z (observed): 187,218, m/z (calculated): 188,376).

From the above experimental results, the associated product of the present invention was found to include a trimer and a hexamer.

### [Example 1] a Vaccine Containing a Molecular Needle Carrying a Test Peptide which is Conceived to be an MHC Class II Peptide of RS Virus

In Example 1, the vaccine of the present invention containing a molecular needle carrying a test peptide as an active ingredient was prepared. The test peptide was formed of 68 amino acid residues of L protein of RSV. The effect of the prepared vaccine was investigated. As a result, the vaccine was found to serve as the vaccine 3 of the present invention, exhibiting not only class II function but also class I function.

### (a) Production Example

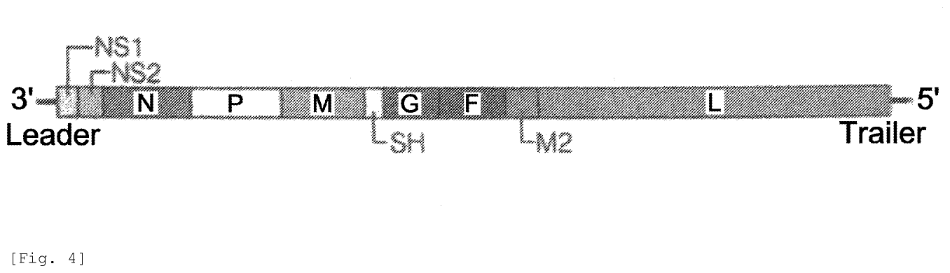

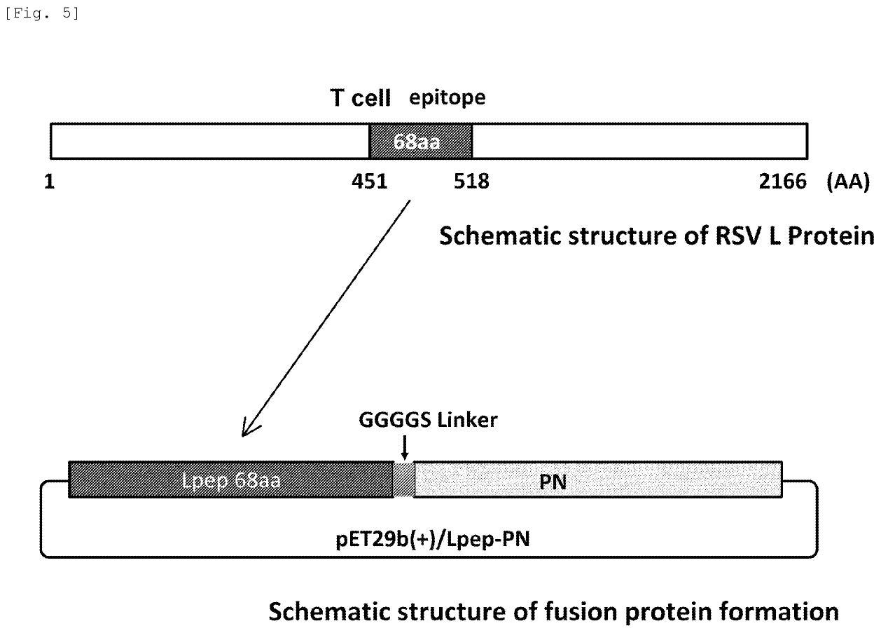

L protein of RSV is an RNA polymerase formed of 2, 166 amino acid residues (GenBank Ref.: KM517573, SEQ ID NO: 23) and a non-structural protein synthesized in the infected cells (FIG. 4).

The amino acid sequence (each amino acid being represented by a single letter abbreviation) is as follows:

“MDPIINGSSANVYLTDSYLKGVISFSECNALGSYLENGPYLKND
YTNLISRQSPLIEHMNLKKLTITQSLISRYHKGELKLEEPTYFQS
LLMTYKSMSSSEQIATTNLLKKIIRRAIEISDVKVYAILNKLGLK
EKDRVKPNNNSGDENSVLTTIIKDDILSAVENNQSYTNSDKNYSV
NQNINIKTTLLKKLMCSMQHPPSWLIHWFNLYTKLNNILTQYRSN
EVKSHGFILIDNQTLSGFQFILNQYGCIVYHKGLKKITTTTYNQF
LTWKDISLSRLNVCLITWISNCLNTLNKSLGLRCGFNNVVLSQLF
LYGDCILKLFHNEGFYIIKEVEGFIMSLILNITEEDQFRKRFYNS
MLNNITDAAIKAQKDLLSRVCHILLDKTVSDNIINGKWIILLSKF
LKLIKLAGDNNLNNLSELYFLFRIFGHPMVDERQAMDAVRINCNE
TKFYLLSSLSTLRGAFIYRIIKGFVNTYNRWPTLRNAIVLPLRWL
NYYKLNTYPSLLEITENDLIILSGLRFYREFHLPKKVDLEMIIND
KAISPPKDLIWTSFPRNYMPSHIQNYIEHEKLKFSESDRSRRVLE
YYLRDNKFNECDLYNCVVNQSYLNNSNHVVSLTGKERELSVGRMF
AMQPGMFRQIQILAEKMIAENILQFFPESLTRYGDLELQKILELK
AGISNKSNRYNDNYNNYISKCSIITDLSKFNQAFRYETSCVCSDV
LDELHGVQSLESWLHLTIPLVTIICTYRHAPPFIKDHVVNLNEVD
EQSGLYRYHMGGIEGWCQKLWTIEAISLLDLISLKGKESITALIN
GDNQSIDISKPVRLIEGQTHAQADYLLALNSLKLLYKEYAGIGHK
LKGTETYISRDMQFMSKTIQHNGVYYPASIKKVLRVGPWINTILD
DFKVSLESIGSLTQELEYRGESLLCSLIFRNIWLYNQIALQLRNH
ALCNNKLYLDILKVLKHLKTFFNLDSIDTALSLYMNLPMLFGGGD
PNLLYRSFYRRTPDFLTEAIVHSVFVLSYYTGHDLQDKLQDLPDD
RLNKFLTCVITEDKNPNAEFVTLMRDPQALGSERQAKITSEINRL
AVTEVLSIAPNKIFSKSAQHYTTTEIDLNDIMQNIEPTYPHGLRV
VYESLPFYKAEKIVNLISGTKSITNILEKTSAIDTTDINRATDMM
RKNITLLIRILPLDCNKDKRELLSLENLSITELSKYVRERSWSLS
NIVGVTSPSIMFTMDIKYTTSTIASGIIIEKYNVNGLTRGERGPT
KPWVGSSTQEKKTMPVYNRQVLTKKQRDQIDLLAKLDWVYASIDN
KDEFMEELSTGTLGLSYEKAKKLFPQYLSVNYLHRLTVSSRPCEF
PASIPAYRTTNYHEDTSPINHVLTEKYGDEDIDIVFQNCISFGLS
LMSVVEQFTNICPNRIILIPKLNEIHLMKPPIFTGDVDIIKLKQV
IQKQHMFLPDKISLTQYVELFLSNKALKSGSHINSNLILVHKMSD
YFHNAYILSTNLAGHWILIIQLMKDSKGIFEKDWGEGYITDHMFI
NLNVFFNAYKTYLLCFHRGYGKAKLECDMNTSDLLCVLELIDSSY
WKSMSKVFLEQKVIKYIVNQDTSLHRIKGCHSFKLWELKRLNNAK
FTVCPWVVNIDYHPTHMKAILSYIDLVRMGLINVDKLTIKNKNKE
NDEFYTSNLFYISYNFSDNTHLLTKQIRIANSELEDNYNKLYHPT
PEALENISSIPVKSNNRNKPKFCISGSTESMMTSTESNKMHIKSS
TVTTRFNYSRQDLYNLFPIVVIDRIIDHSGNTEKSNQLYTTTSHQ
TSLVRNSASLYCMLPWHHVNRENFVFSSTGCKISIEYILKDLKIK
DPSCIAFIGEGAGNLLLRTVVELHPDIRYIYRSLKDCNDHSLPIE
FLRLYNGHINIDYGENLTIPATDATNNIHWSYLHIKFAEPISIFV
CDAELPVTANWSKIIIEWSKHVRKCKYCSSVNRCILIAKYHAQDD
IDFKLDNITILKTYVCLGSKLKGSEVYLVLTIGPANILPVFDVVQ
NAKLILSRTKNFIMPKKIDKESIDANIKSLIPFLCYPITKNGIKT
SLSKLKSVVNGDILSYSIAGRNEVESNKLINHKHMNILKWLDHVL
NERSAELNYNHLYMIESTYPYLSELLNSLTTNELKKLIKITGSVL
YNLPNEQ.”

A domain of L protein from the 451th to the 518th amino acid residues (68 amino acid residues in total) (SEQ ID NO: 24) is conceived to be an epitope recognized by an MHC II receptor molecule of T cells (i.e., an MHC class II peptide). The domain binds to P protein (phosphorylated protein: a non-structural protein of RSV involved in phosphorylation).

The above sequence of 68 amino acid residues (SEQ ID NO: 24) is as follows (each amino acid being represented by a single letter abbreviation): “KFYLLSSLSTLRGAFIYRIIKGFVNTYNRWPTLRNAIVLPLRWLNYYKLNTYPSLLEITE NDLIILSG.” In Example 1, a molecular needle carrying the sequence as W in formula (1) was produced.

All the reagents employed in Example 1 were purchased from commercial suppliers and used without additional purification. The gene fragment of L protein of RSV was obtained from the genome RNA of RSV-Long variant provided by Sawada, Kitasato Institute for Life Science & Graduated School of Infection control Science I. Specifically, a terminal stop codon was removed from the L protein gene sequence, and the resultant part was amplified. In addition, a gene fragment encoding the peptide domain of L protein from the 451th to the 518th amino acid residues was obtained by amplifying the same fragment.

Then, a plasmid (pET29b(+)/F-PN), which was designed to fuse with PN by the mediation of “GGGGS” (SEQ ID NO: 15) at the C-terminal side of the peptide formed of 68 amino acid residues, was created. E. coli (BL21 DE3) cells were transformed with the plasmid, and expression was induced by IPTG.

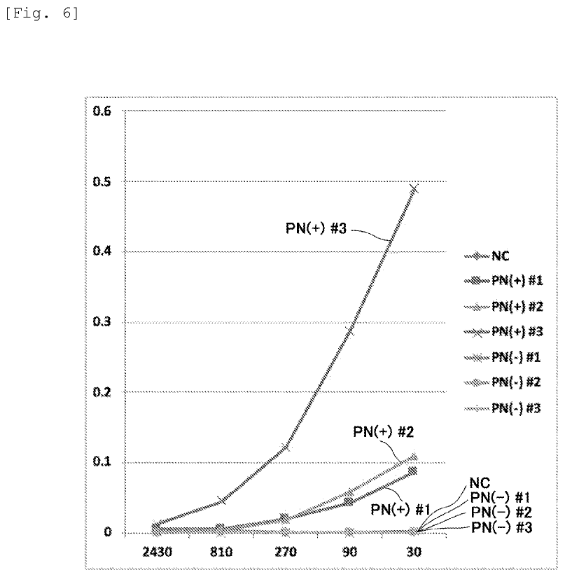

In this example, the plasmid structure “PN-FL-VPg” produced in “(b)-3” above was used as a template. Through In-Fusion cloning, Vpg was substituted with the gene fragment encoding the peptide including the MHC class I peptide, to thereby create a plasmid structure of interest “pET29b (+)/Lpep-PN.” (FIG. 5).

In a more specific procedure, a peptide domain of ATG (M)-added L protein (the 451th to the 518th amino acid residues) was amplified by the mediation of In-Fusion RS-P sense

(SEQ ID NO: 25)
5′-GGAGATATACATATGaagttctatttattaagtag-3′

and In-Fusion RS-P antisense

(SEQ ID NO: 26)
5′-TGAACCCCCGCCTCCtcctgataaaataatcaaatc-3′,

to thereby yield a P domain amplification product to which the underlined portions had been added. Separately, inverted PCR was performed by use of 5′-GGAGGCGGGGGTTCA-3′ (SEQ ID NO: 27) and 5′-ATGTATATCTCCTTCTTAAAG-3′ (SEQ ID NO: 28) as primers with the plasmid structure “PN-FL-VPg” as a template, to thereby amplify the entirety of the vector portion excepting the sequence of VPg. Thus, a vector body was provided. These two fragments were linked through In-Fusion cloning, to thereby yield a plasmid structure of interest “pET29b (+)/Lpep-PN.”

Subsequently, the above plasmid was introduced into DH5α-competent cells. The thus-obtained vector was identified through DNA nucleotide sequencing and then, Lpep-PN(+) was formed through gene expression.

In the gene expression, E. Coli BL21 (DE3) having the “pET29b (+)/Lpep-PN” plasmid was cultured overnight in an LB medium containing 30 μg/mL Kanamycin at 37° C. After the OD₆₀₀ of the incubated product solution (37° C.) had reached 0.8, 1 mM isopropyl-β-D-1-thiogalactopyranoside (IPTG) and arabinose were added to the culture product. 16 to 17 hours after addition of IPTG and arabinose, the culture product was centrifuged at 8,000 rpm for 5 minutes, to thereby recover microorganism pellets, and the recovered matter was incubated at 20° C. and a rotation rate of 180 rpm. Subsequently, in the presence of ice, the cell pellets were suspended in 100 mM Tris-HCl (pH: 8.0) buffer containing 5 mM imidazole with one tablet of “complete, EDTA-free,” and the cells were lysed through ultrasonication. Cell broken pieces were removed through centrifugation (17,500 rpm for 50 minutes). The supernatant was filtered through a 0.8-μm filter, and the filtrate was added to an Ni affinity column. The added matter was eluted at 4° C. with the same buffer under a linear imidazole concentration gradient of 5 mM to 250 mM. Thereafter, the Lpep-PN associated product was dialyzed against 20 mM Tris/HCl (pH: 8.0) with 0.2M NaCl and further against PBS, and the dialysis product was concentrated through ultrafiltration. Through this process, Lpep-PN spontaneously yielded an associated product containing a trimer and/or a hexamer thereof. The thus-obtained product was employed in immunoassay as a “Lpep-PN associated product.” As a control, “a peptide including an MHC class I peptide derived from L protein and formed of 68 amino acid residues” was prepared through a customary procedure including a gene amplification technique.

### (b) Immunoassay (1) of Example 1 (Antibody Titer Measurement)

Each of the Lpep-PN associated product group (PN(+)) and the test peptide derived from L protein and formed of 68 amino acid residues (hereinafter may be referred to as an LMHC peptide) group (PN(−)) was nasally inoculated to RS virus-susceptible cotton rats (3 rats per group). In all nasal inoculation cases, a vaccine liquid was added dropwise to the nasal cavity, and a purified antigen was caused to be inhaled at 300 μL (20 μg) per rat through the nasal cavity under anesthesia. The nasal inoculation included initial inoculation and booster inoculations (twice) at intervals of 7 days. Blood collection was performed before the initial inoculation, 1 week after the initial inoculation (before the first booster), 2 weeks after the initial inoculation (before the second booster), and 3 weeks after the initial inoculation. Each blood sample was caused to react with L protein of RSV, and the IgG titer and the IgA titer to the L protein of RSV in serum were determined through ELISA.

Seven weeks after the initial inoculation, the final booster inoculation was conducted.

In a specific procedure of ELISA, the antigen (RSV L protein) was diluted with PBS (−) to 2 μg/mL. The diluted antigen was added to a 96-well ELISA plate at 50 μL/well and incubated overnight at 4° C. The plate was washed thrice with PBST (0.1% Tween 20/PBS) and then, PBSB (1% BSA/PBS) was added to the plate at 80 μL/well. Incubation was further conducted at room temperature for 2 hours for blocking. Subsequently, a test serum sample was diluted with PBSB, to thereby prepare test samples (for IgG detection: 5-fold dilution series (10-fold to 31, 250-fold), and for IgA detection: 3-fold dilution series (10-fold to 2, 430-fold). PBSB in the plate was removed, and each test sample was added to the plate at 50 μL/well. Incubation was performed at room temperature for 2 hours, and the plate was washed 5 times with PBST. Then, an HRP substrate solution was added to the plate at 50 μL/well. Incubation was performed at room temperature under light shielding conditions until color development was confirmed. The reaction was stopped by adding 2M sulfuric acid to the plate at 25 μL/well, and absorbance at 490 nm was measured.

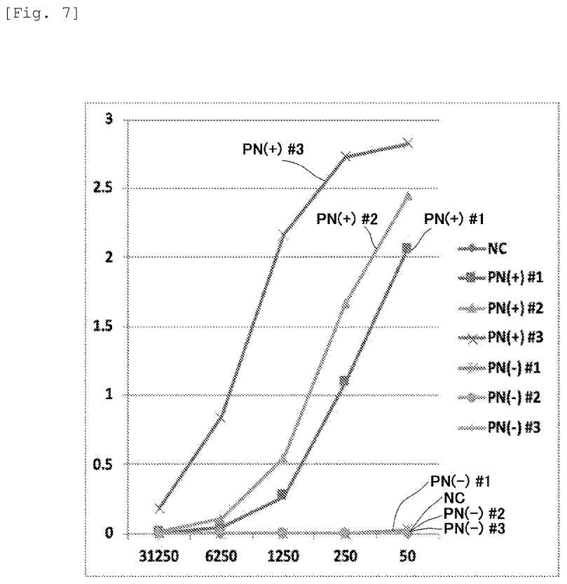

FIG. 6 shows the results of ELISA (IgA), and FIG. 7 shows the results of ELISA (IgG), obtained 3 weeks after the initial inoculation (3W). In each graph, the vertical axis represents absorbance, and the horizontal axis represents serum dilution ratio. The graphs show the results of the cotton rats tested under different conditions. As shown in the graphs, when the rats were immunized directly with LMHC peptide, no rise in absorbance in response to antibody titer was observed in both cases of IgG and IgA. In contrast, when immunization was conducted with the associated product of molecular needles carrying the test peptide, the antibody titers (i.e., absorbance values) were found to rise remarkably, indicating considerable induction of humoral immunity. Furthermore, it was noteworthy that such a remarkable rise in antibody titer was attained by use of no adjuvant. Therefore, the associated product of the present invention can possibly be used as an adjuvant-free vaccine.

An RS virus infection is caused by inhaling the RS virus via droplet infection. That is, the infection is mainly caused via the oral cavity or the nasal cavity. Thus, the associated product was introduced directly into the nasal mucosal cells through the cell membrane by nasal inoculation, whereby humoral immunity in the nasal cavity was induced in the nasal mucosal cells, and protective immunity to RS virus was elicited. In this way, when a molecular needle carrying a test peptide was nasally inoculated as an immunogen, topical immunity was found to be induced. Particularly, induction of IgA to L protein, which is a non-structural protein of RS virus, is expected to inhibit the function of L protein through a mechanism including binding to the L protein upon secretion of IgA produced in cells into the mucous layer. In addition, a domain to which the induced antibody is bound also serves as a domain which binds to P protein for exhibiting the function of L protein. Therefore, conceivably, inhibition of formation of an L protein-P protein complex may lead to more efficient inhibition of replication/proliferation of the target virus.

### (c) Immunoassay (2) of Example 1 (Lung Inflammatory Inhibiting Effect)

In the immunoassay (2), RS virus-susceptible cotton rats were immunized with a molecular needle carrying a test peptide, and the rats were tested. Through employment of the rats, the effect of inhibiting lung inflammation caused by RS virus infection was directly investigated.

Two weeks after the last booster inoculation carried out 7 weeks after the initial inoculation in the aforementioned immunoassay (1) (i.e., 9 weeks after the initial inoculation), the test cotton rats were infected with an RS virus (Long variant) with an infection dose of 2×10⁵ PFU/mL. The infection with an RS virus was conducted in the same manner (nasal inoculation) as employed in the vaccination. In the infection experiment, a non-immunized/infected rat group (Not immunized: 3 rats) was added as a positive control.

Four days after the above infection, a lung tissue was recovered from each tested rat, and the amount of RS virus in the lungs was determined. In a specific procedure, the lung, being an infection target tissue, was removed and processed by a homogenizer. The product was suspended in a medium, to thereby release the relevant virus, and the amount of infectious virus contained in the lung tissue (50 mg) was determined through plaque assay.

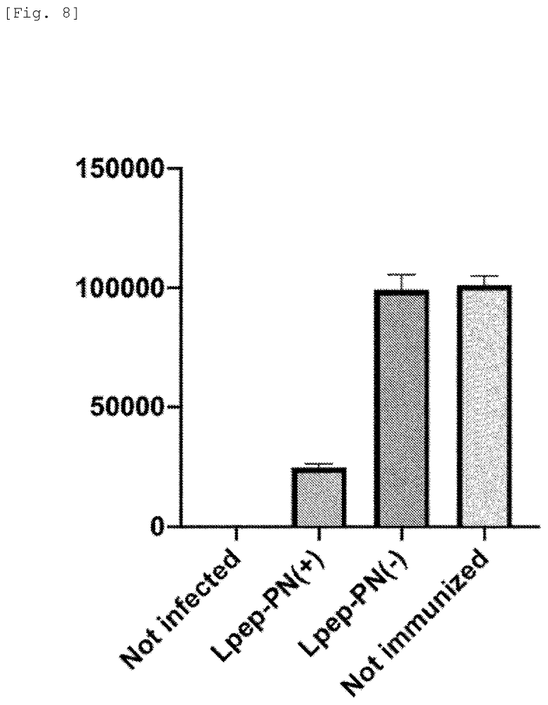

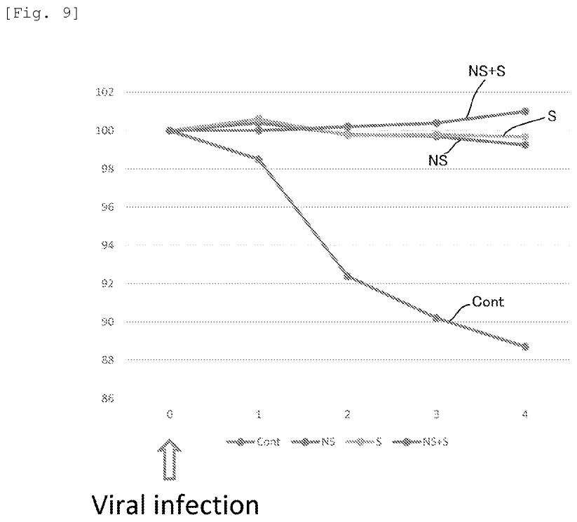

FIG. 8 shows the results. On the horizontal axis of the graph of FIG. 8, “Not infected” represents the negative control (n=1), “Lpep-PN(+)” an Lpep-PN associated product group (n=3), “Lpep-PN(−)” an LMHC peptide group (n=3), and “Not immunized” the positive control (n=3). The vertical axis represents the RS virus titer (PFU/50 mg-lung tissue) of the recovered lung tissue. As is clear from FIG. 8, RS virus infection of rats to which the Lpep-PN associated product had been administered was found to be inhibited at about 65% with a significance of (P<0.0001), as compared with the case of RS virus-infected rats (i.e., a positive control group).

Conceivably, the remarkable effect was achieved by the test peptide attached to the molecular needle. Specifically, the peptide induced not only the aforementioned humoral immunity but also cell-mediated immunity, to thereby remarkably effectively reduce the amount of the RS virus in the lungs. Therefore, it is highly likely that the test peptide which was initially estimated to include an MHC class II recognition motif also includes an MHC class I recognition motif. This is because such a strong suppressive effect on the amount of the RS virus cannot be explained solely by the humoral immunity response, but it is thought that the effect of eliminating virus-infected cells by induction of cell-mediated immunity is exerted.

### [Example 2] Studies on Novel Coronavirus

### (1) General View of Novel Coronavirus

A novel coronavirus (SARS-COV-2) (hereinafter may be referred to simply as “a novel coronavirus”) belongs to the immunogenic group 2 of the genus Coronavirus (β-coronavirus), similar to SARS coronavirus, which is a pathogen of SARS (severe acute respiratory syndrome); a human coronavirus OC43 variant also causing an upper respiratory infection in humans; and a human coronavirus HKU1 variant causing lower respiratory infection in humans. Such infections are also observed in mice, cows, pigs, etc.

Similar to other viruses belonging to the genus Coronavirus, the novel coronavirus assumes a spherical particle having a diameter of some 100 nanometers, and having petal-shaped spikes with a narrow base and an expanded tip. The structural protein of the novel coronavirus includes, in the envelope thereof, a spike protein (S), a membrane protein (M), and an envelope protein (E). The S protein is a glycoprotein whose trimer forms one petal-shaped spike and has adsorbability to a virus receptor (i.e., an angiotensin-converting enzyme II (ACE2)) present in the host cells, and a membrane fusing ability by the mediation of the action of a serine protease (TMPRSS2). The S protein may be also a target of immune response of the host, as a neutralization epitope or a T-cell epitope. Each of M protein and E protein is also a glycoprotein, which is mostly present in the lipid bilayer and plays an important role in forming virus particles.

N (nucleocapsid) protein is an RNA-binding phosphorylated protein. N protein binds to viral genomic RNA, to thereby form a nucleocapsid, and is involved in replication, transcription, and translation of the RNA.

The genome of a novel coronavirus is positive-sense single-stranded RNA. The genome per se functions as mRNA and is infectious. At least similar to SARS coronavirus, the 5′-terminal of the genome has a cap structure, and the 3′-terminal has poly A. At the 5′-terminal, a leader sequence which modulates gene replication and transcription and an untranslated region are present. In the downstream thereof, a non-structural protein gene encoding an enzyme (replicase) (e.g., RNA polymerase or protease) essential for proliferation of a virus and structural genes encoding the aforementioned S, E, M, and N proteins exist.

More specifically, the aforementioned S protein is formed of 1,273 amino acids and includes SS (signal sequence), NTD (N-terminal domain), RBD (receptor-binding domain), SD1 (subdomain 1), SD2 (subdomain 2), S1/S2 (S1/S2 protease cleavage site), S2′ (S2′ protease cleavage site), FP (fusion peptide), HR1 (heptad repeat 1), CH (central helix), CD (connector domain), HR2 (heptad repeat 2), TM (transmembrane domain), and CT (cytoplasmic tail). RBD is formed of a trimer formed from two down-state protomers and one up-state protomer (see, for example, Non-Patent Documents 1 and 2).

The non-structural protein is not a protein forming particles of a novel coronavirus, but is a protein in relation to replication/proliferation of the virus. The protein is synthesized in cells only through a series of virus protein formation steps including adhesion and invasion of the virus into the cells and introduction of the virus genome into the cells. In the synthesis of a novel coronavirus protein, only an ORF at the 5′-terminal of each mRNA is translated. Two big ORFs (i.e., ORF1a and ORF1b) are present between the ORF of mRNA-1 and the ORF of mRNA-2, and are translated to Proteins of 1a and 1b, respectively, whereby two proteins of 1a and 1a+1b are synthesized. Protein 1a is cleaved into non-structural proteins (nsp-1 to nsp-11) by proteases included therein (i.e., nsp-3 (non-structural composite protein-3, papain-like proteases) and nsp-5 (main protease)). Protein 1a+1b is cleaved into non-structural proteins (nsp-1 to nep-10) and in addition non-structural proteins (nsp-12 to nep-16) during or after translation. RNA-dependent RNA polymerase (nsp-12) and helicase (nsp-13) are produced as cleavage products from the domain 1b. These various non-structural proteins are required for proliferation of a virus. Each of “ORF3a,” “ORF6,” “ORF7a,” and “ORF8,” which are shown in Table 1 below as the peptides derived from non-structural proteins, is an open reading frame which is present in the most downstream region (after the 25,000th base in the genome).

### (2) Test Peptides Employed in Example 2

In Example 2, each of the peptides formed of 9 amino acid residues shown in Table 1 was employed as a test peptide (W), and, in a manner similar to that of Example 1, the peptide was attached to form a composite protein, and molecules of the composite protein were associated to prepare a molecular needle (hexamer). In Table 1, “Base position” is given in reference to “GenBank accession No. MN908947.” As an exceptional case, RBD (receptor binding domain protein) was used as a test protein. All the reagents employed in Example 2 were purchased from commercial suppliers and used without additional purification.

RBD forms a part of S protein, which is a structural protein of a novel coronavirus (SARS-COV-2). RBD protein is encoded as a template sequence of mRNA of the S protein in the genome of the novel coronavirus.

The amino acid sequence of the actually employed RBD protein of the novel coronavirus (SARS-COV-2) serving as an immunogen corresponds to an underline portion (310aa-540aa) of an amino acid sequence of S protein encoded by the bases 21563 to 25384 of the spike gene (S-gene) of prototype SARS-CoV-2 GenBank accession No. MN908947. The amino acid sequence of S protein is represented by

SEQ ID NO: 29:
“MFVFLVLLPLVSSQCVNLTTRTQLPPAYTNSFTRGVYYPDKVFR
SSVLHSTQDLFLPFFSNVTWFHAIHVSGTNGTKRFDNPVLPENDG
VYFASTEKSNIIRGWIFGTTLDSKTQSLLIVNNATNVVIKVCEFQ
FCNDPFLGVYYHKNNKSWMESEFRVYSSANNCTFEYVSQPFLMDL
EGKQGNFKNLREFVFKNIDGYFKIYSKHTPINLVRDLPQGFSALE
PLVDLPIGINITRFQTLLALHRSYLTPGDSSSGWTAGAAAYYVGY
LQPRTFLLKYNENGTITDAVDCALDPLSETKCTLKSFTVEKGIYQ
TSNFRVQPTESIVRFPNITNLCPFGEVENATRFASVYAWNRKRIS
NCVADYSVLYNSASFSTFKCYGVSPTKLNDLCFTNVYADSFVIRG
DEVRQIAPGQTGKIADYNYKLPDDFTGCVIAWNSNNLDSKVGGNY
NYLYRLFRKSNLKPFERDISTEIYQAGSTPCNGVEGENCYFPLQS
YGFQPTNGVGYQPYRVVVLSFELLHAPATVCGPKKSTNLVKNKCV
NFNENGLTGTGVLTESNKKFLPFQQFGRDIADTTDAVRDPQTLEI
LDITPCSFGGVSVITPGTNTSNQVAVLYQDVNCTEVPVAIHADQL
TPTWRVYSTGSNVFQTRAGCLIGAEHVNNSYECDIPIGAGICASY
QTQTNSPRRARSVASQSIIAYTMSLGAENSVAYSNNSIAIPTNFT
ISVTTEILPVSMTKTSVDCTMYICGDSTECSNLLLQYGSFCTQLN
RALTGIAVEQDKNTQEVFAQVKQIYKTPPIKDFGGFNFSQILPDP
SKPSKRSFIEDLLENKVTLADAGFIKQYGDCLGDIAARDLICAQK
FNGLTVLPPLLTDEMIAQYTSALLAGTITSGWTFGAGAALQIPFA
MQMAYRENGIGVTQNVLYENQKLIANQFNSAIGKIQDSLSSTASA
LGKLQDVVNQNAQALNTLVKQLSSNFGAISSVLNDILSRLDKVEA
EVQIDRLITGRLQSLQTYVTQQLIRAAEIRASANLAATKMSECVL
GQSKRVDFCGKGYHLMSFPQSAPHGVVFLHVTYVPAQEKNFTTAP
AICHDGKAHFPREGVFVSNGTHWFVTQRNFYEPQIITTDNTFVSG
NCDVVIGIVNNTVYDPLQPELDSFKEELDKYFKNHTSPDVDLGDI
SGINASVVNIQKEIDRLNEVAKNLNESLIDLQELGKYEQYIKWPW
YIWLGFIAGLIAIVMVTIMLCCMTSCCSCLKGCCSCGSCCKFDED
DSEPVLKGVKLHYT,”

the underlined portion being “KGIYQTSNFRVQPTESIVRFPNITNLCPFGEVFNATRFASVYAWNRKRISNCVADYSVLY NSASFSTFKCYGVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPCNGVEGENCYFP LOSYGFQPTNGVGYQPYRVVVLSFELLHAPATVCGPKKSTNLVKNKCVN” (SEQ ID NO: 30). The nucleic acid sequence encoding the above may be selected in accordance with a generally known amino acid-nucleobase relationship.

In Example 2, an internal sequence 22,491 to 23,182: “aaggaatctatcaaacttctaactttagagtccaaccaacagaatctattgttagatttc ctaatattacaaacttgtgcccttttggtgaagtttttaacgccaccagatttgcatctgt ttatgcttggaacaggaagagaatcagcaactgtgttgctgattattctgtcctatataat tccgcatcattttccacttttaagtgttatggagtgtctcctactaaattaaatgatctct gctttactaatgtctatgcagattcatttgtaattagaggtgatgaagtcagacaaatcgc tccagggcaaactggaaagattgctgattataattataaattaccagatgattttacaggc tgcgttatagcttggaattctaacaatcttgattctaaggttggtggtaattataattacc tgtatagattgtttaggaagtctaatctcaaaccttttgagagagatatttcaactgaaat ctatcaggccggtagcacaccttgtaatggtgttgaaggttttaattgttactttccttta caatcatatggtttccaacccactaatggtgttggttaccaaccatacagagtagtagtac tttcttttgaacttctacatgcaccagcaactgtttgtggacctaaaaagtctactaattt ggttaaaaacaaatgtgtcaat” (SEQ ID NO: 31) in the bases 21, 563 to 25, 384 of the spike gene (S-gene) MN908947 was employed.

The gene fragment “TTTCGTGTTCAGCCGACCGAAAGCATTGTTCGTTTTCCGAATATCACCAATCTGTGTCCG TTTGGCGAAGTTTTTAATGCAACCCGTTTTGCAAGCGTTTATGCCTGGAATCGTAAACGTA TTAGCAATTGCGTTGCCGATTATAGCGTTCTGTATAATAGCGCAAGCTTCAGCACCTTTAA ATGCTATGGTGTTAGCCCGACCAAACTGAATGATCTGTGTTTTACCAATGTGTATGCCGAT AGCTTTGTGATTCGTGGTGATGAAGTTCGTCAGATTGCACCGGGTCAGACCGGTAAAATTG CAGATTATAACTATAAACTGCCGGATGATTTTACGGGTTGTGTTATTGCATGGAATAGCAA TAACCTGGATAGCAAAGTTGGTGGCAACTATAACTATCTGTATCGCCTGTTTCGTAAGAGC AATCTGAAACCGTTTGAACGTGATATTAGCACCGAAATTTATCAGGCAGGTAGCACCCCGT GCAATGGTGTTGAAGGTTTTAATTGTTATTTTCCGCTGCAGAGCTATGGTTTTCAGCCTAC CAATGGTGTGGGTTATCAGCCGTATCGTGTTGTTGTTCTGTCATTTGAACTGCTGCATGCA CCGGCAACCGTT” (SEQ ID NO: 32) corresponding to an amino acid sequence of “MFRVQPTESIVRFPNITNLCPFGEVFNATRFASVYAWNRKRISNCVADYSVLYNSASFST FKCYGVSPTKLNDLCFTNVYADSFVIRGDEVRQIAPGQTGKIADYNYKLPDDFTGCVIAWN SNNLDSKVGGNYNYLYRLFRKSNLKPFERDISTEIYQAGSTPCNGVEGENCYFPLQSYGFQ PTNGVGYQPYRVVVLSFELLHAPATV” (SEQ ID NO: 33) in the RBD was obtained by amplifying the relevant portion of the gene sequence of S protein through RT-PCR, wherein the gene sequence of S protein was obtained from a virus variant KUH003 (LC630936) D614G separated from infection patients and cultured by Graduate School of Infection Control Science, Ohmura Satoshi Memorial Institute, Kitasato University. To the gene fragment, 5′ primer ggagatatacatatg sequence (SEQ ID NO: 34) for RT-PCR and 3′ primer ggaggcgggggttca sequence (SEQ ID NO: 35) (corresponding to a GGGGS linker) were attached for In-Fusion cloning, and the resultant fragment was used for producing a molecular needle.

In Table 1 below, the reference “S” in the left-upper section denotes a peptide derived from a structural protein (RBD: a protein as mentioned above), and the reference “NS” in the left-lower section denotes a peptide derived from a non-structural protein.

TABLE 1

| | Sample | | | Amino | | |
|---|---|---|---|---|---|---|
| | number | | | acid | | |
| | ↓ | Plasmid | Protein | position | Amido acid sequence | Base position*⁾ |
| S | #3 | S109 | S | 109-117 | TLDSKTQSL (SEQ ID NO: 36) | 21887-21913 |
| | #1 | S269 | S | 269-277 | YLQPRTFLL (SEQ ID NO: 37) | 22367-22393 |
| | RBD | S456 | S | RBD domain | — | — |
| | #4 | S680 | S | 680-688 | SPRRARSVA (SEQ ID NO: 38) | 23600-23626 |
| | #2 | S976 | S | 976-984 | VLNDILSRL (SEQ ID NO: 39) | 24488-24514 |
| | #5 | E50 | E | 50-58 | SLVKPSFYV (SEQ ID NO: 40) | 26392-26418 |
| | #6 | M15 | M | 15-23 | KLLEQWNLV (SEQ ID NO: 41) | 26565-26591 |
| | #21 | M108 | M | 108-116 | SMWSFNPET (SEQ ID NO: 42) | 26844-26870 |
| | #14 | M164 | M | 164-172 | LPKEITVAT (SEQ ID NO: 43) | 27012-27038 |
| | #8 | N66 | N | 66-74 | FPRGQGVPI (SEQ ID NO: 44) | 28469-28495 |
| | #10 | N105 | N | 105-113 | SPRWYFYYL (SEQ ID NO: 45) | 28586-28612 |
| | #7 | N222 | N | 222-230 | LLLDRLNQL (SEQ ID NO: 46) | 28937-28963 |
| | #9 | N257 | N | 257-265 | KPRQKRTAT (SEQ ID NO: 47) | 29042-29068 |
| | #15 | N338 | N | 338-346 | KLDDKDPNE (SEQ ID NO: 48) | 29285-29311 |
| NS | #16 | ORF3a-35 | ORF3a | 35-43 | IPIQASLPF (SEQ ID NO: 49) | 25495-25521 |
| | #12 | ORF3a-72 | ORF3a | 72-80 | ALSKGVHFV (SEQ ID NO: 50) | 25606-25632 |
| | #17 | ORF3a-103 | ORF3a | 103-111 | APFLYLYAL (SEQ ID NO: 51) | 25699-25725 |
| | #11 | ORF3a-139 | ORF3a | 139-147 | LLYDANYFL (SEQ ID NO: 52) | 25807-25833 |
| | #13 | ORF6-3 | ORF6 | 3-11 | HLVDFQVTI (SEQ ID NO: 53) | 27208-27234 |
| | #22 | ORF7a-4 | ORF7a | 4-12 | ILFLALITL (SEQ ID NO: 54) | 27403-27429 |
| | #19 | ORF7a-78 | ORF7a | 78-86 | RARSVSPKL (SEQ ID NO: 55) | 27625-27651 |
| | #18 | ORF7a-85 | ORF7a | 85-93 | KLFIRQEEV (SEQ ID NO: 56) | 27646-27672 |
| | #20 | ORF8-73 | ORF8 | 73-81 | YIDIGNYTV (SEQ ID NO: 57) | 28110-28136 |

*⁾With reference to GenBank accession No. MN908947

### (3) Immunoassay 1

In the assay system 1 of Example 2, Syrian hamsters were immunized in a predetermined manner, and then infected with a novel coronavirus. After infection, the change in body weight and the amount of virus in the lungs were monitored.
(a) The following five test animal groups each consisting of 3 Syrian hamsters were prepared.

S-PN(+): Test vaccine administration group (Group 1). The administered test vaccine was prepared as follows: A mixture prepared by mixing, in equal mass, molecular needles (hexamers) carrying each of the 13 structural protein peptides and RBD shown in Table 1 was added to physiological saline to prepare the test vaccine so that one dosage unit per hamster was 40 μg of the molecular needles (in 30 mL of physiological saline).

NS-PN(+): Test vaccine administration group (Group 2). The administered test vaccine was prepared as follows: A mixture prepared by mixing, in equal mass, molecular needles (hexamers) carrying each of 9 non-structural protein peptides shown in Table 1 was added to physiological saline to prepare the test vaccine so that one dosage unit per hamster was 40 μg of the molecular needles (in 30 mL of physiological saline).

S-PN(+) & NS-PN(+): Test vaccine administration group (Group 3). The administered test vaccine was prepared as follows: A mixture (40 μg) prepared by mixing, in equal mass, molecular needles carrying each of the 13 structural protein of S-PN(+) and RBD and a mixture (40 μg) prepared by mixing, in equal mass, molecular needles carrying each of the 9 non-structural protein of NS-PN(+) were added to physiological saline to prepare the test vaccine so that one dosage unit was a total of 80 μg of the molecular needles in 30 mL of physiological saline.
SARS-COV-2 inf: SARS-COV-2 infection group (Group 4).
NC: Non-immunized (Non-infected) group.
(b) The novel coronavirus used in the infection step was a virus variant KUH003 (LC630936) D614G separated from infection patients and cultured by Graduate School of Infection Control Science, Ohmura Satoshi Memorial Institute, Kitasato University.
(c) Assay system 1

In immunoassay 1, each of the above molecular needles carrying test peptides was administered to the Syrian hamsters of Groups 1 to 3. Administration was carried out in respective dosage units through nasal inoculation. In all nasal inoculation cases, a vaccine liquid was added dropwise to the nasal cavity and was caused to be inhaled at 300 μL (40 μg) per hamster through the nasal cavity under anesthesia. The nasal inoculation included initial inoculation and booster inoculations (twice) at intervals of 7 days. Blood collection was performed before the initial inoculation, 1 week after the initial inoculation (before the booster inoculation), 2 weeks after the initial inoculation (before the second booster inoculation), and 3 weeks after the initial inoculation. Then, seven weeks after the initial inoculation, blood collection was performed and then the final booster inoculation was administered. Two weeks after the final booster inoculation, each test hamster was infected with the novel coronavirus. The infection dose of the novel coronavirus was 2×10⁵ PFU/mL. The infection with the novel coronavirus was conducted in the same manner (nasal inoculation) as employed in the vaccination. In the infection experiment, a non-immunized/infected hamster group was added as a positive control.

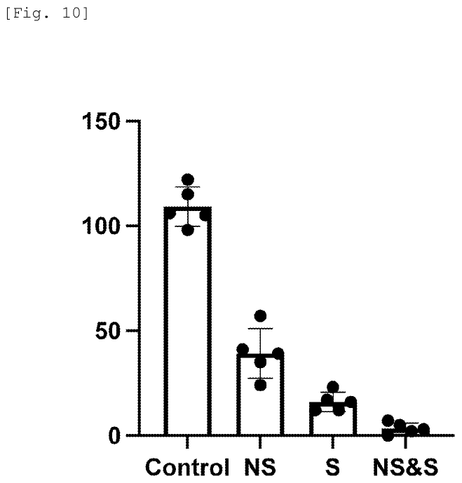

For a period of 4 days after the above infection, the body weight of each test hamster was measured, and the change in body weight attributed to infection with the novel coronavirus was monitored. FIG. 9 shows the change in body weight. Also, on the final day 4, a lung tissue was recovered from each tested hamster, and the amount of infection of the novel coronavirus in the lungs was determined. In a specific procedure, the lung, being an infection target tissue, was removed and processed by a homogenizer. The product was suspended in a medium, to thereby release the relevant virus, and the amount of novel coronavirus contained in the lung tissue (50 mg) was determined through plaque assay. FIG. 10 shows the amount of novel coronavirus contained in the lung tissue in each case.

In the graph of FIG. 9, the horizontal axis represents the number of days elapsed, and the vertical axis represents the change in body weight (as a value relative to the initial measurement as 100). The relative values shown are mean values for each group. In Group 1 (S-PN(+)) and Group 2 (NS-PN(+)), no body weight loss was observed. In Group 3 (S-PN(+) & NS-PN(+)), a slight increase in body weight was observed. In Group 4, a considerable body weight loss was observed. The body weight loss observed in Group 4, which received no particular treatment, was found to occur due to reduced appetite and physical exhaustion after infection with the novel coronavirus. In contrast, the initial physical conditions were found to be almost maintained in Group 1 (with administration of S-PN(+)) and Group 2 (with administration of NS-PN(+)). Furthermore, in Group 3 (with administration of two components), a substantially healthy state was found to be maintained.

In the graph of FIG. 10, the vertical axis represents the titer of the novel coronavirus (PFU/50 mg-lung tissue: ×1,000) in the sampled lung tissue. The amount of the virus was found to correlate with the aforementioned loss in body weight. Specifically, in Group 2 (NS-PN(+)), the virus amount was reduced by about 60%. In Group 1 (S-PN(+)), the virus amount was reduced by about 80%. In Group 3 (S-PN(+) & NS-PN(+)), substantially complete disappearance of the virus was observed. The reduction of the virus which was more effective in Group 1 than in Group 2 is conceivably attributed to a strong action of cell-mediated immunity in Group 1. More specifically, most of test peptides in Group I serve as MHC class I peptides effectively inducing cell-mediated immunity, whereas the peptides in Group 2 serve as MHC class II peptides preferentially inducing humoral immunity (supported by the results of immunoassay 2). In Group 3, in which functions of class I and class II were sufficiently attained, proliferation of the novel coronavirus was found to be substantially completely suppressed.

### (4) Immunoassay 2

In immunoassay 2, secretion of a cytokine or chemokine involved in immunity was investigated for each of the molecular needles carrying test peptides shown in Table 1.

The test vaccine administration system was established through the following procedure. Specifically, each of the 13 peptides of structural proteins and RBD, and the 9 peptides of non-structural proteins shown in Table 1 was attached to a molecular needle. Each of the thus-prepared molecular needle was added to physiological saline to prepare a test vaccine so that one dosage unit per mouse was 20 μg of the molecular needle (in 30 mL of physiological saline). A test group consisted of 3 mice.

The schedule of administration of the test vaccine is as follows: To each mouse group, a molecular needle carrying a corresponding test peptide was administered. Administration was carried out in respective dosage units through nasal inoculation. In all nasal inoculation cases, a vaccine liquid was added dropwise to the nasal cavity and was caused to be inhaled at 300 μL (20 μg) per mouse through the nasal cavity under anesthesia. The nasal inoculation included initial inoculation and booster inoculations (twice) at intervals of 7 days. Blood collection was performed before the initial inoculation, 1 week after the initial inoculation (before the booster inoculation), 2 weeks after the initial inoculation (before the second booster inoculation), and 3 weeks after the initial inoculation. Then, seven weeks after the initial inoculation, blood collection was performed, and then the final booster inoculation was administered.

Two weeks after the final booster inoculation, each test mouse was stimulated with a suspension of virus-infected cells (i.e., a virus antigen), and whole blood was collected from the mouse 4 days after the stimulation. Then, the spleen was removed from the mouse, and immunocompetent cells were removed from the spleen. The immunocompetent cells were stimulated with a suspension of cells infected with a virus variant KUH003 (LC630936) D614G separated from infection patients and cultured by Graduate School of Infection Control Science, Ohmura Satoshi Memorial Institute, Kitasato University. Before and after the stimulation, the cytokine or chemokine present in the immunocompetent cells was quantitated by means of Bio-Plex. The quantitation values were averaged in each group. The ratio of the quantitation value after stimulation to the quantitation value before stimulation (after stimulation/before stimulation) was calculated with respect to each detection item (the species of cytokine and chemokine) and each test peptide. In evaluation and selection of test peptides of interest, hypersecretion of cytokine or chemokine attributed to stimulation with the virus, characteristic to MHC class I or class II, was checked. Ratings in the evaluation are as follows. Regarding MHC class I, a test peptides for which 5 or more of 7 cytokines and chemokines (i.e., IFN-γ, IL-2, IL-10, IL-12 (p40), IL-12 (p70), MIP-1a, and TNF-α) had the ratio of the qualification values in excess of 2 was evaluated as an MHC I peptides. Regarding MHC class II, a test peptide for which seven or more of 9 cytokines (i.e., IFN-γ, IL-1b, IL-4, IL-5, IL-6, IL-10, IL-12 (p40), IL-12 (p70), and TNF-α) had the ratio of the qualification values in excess of 2 was evaluated as an MHC II peptide.

In the above test, peptides with sample numbers #1, #2, #3, and #4, and RBD were found to exhibit mainly an MHC class II property. That is, since the 4 peptide samples exhibited a rise in cytokine and chemokine expressed upon stimulation via MHC class II, the peptides were determined as class II peptides. Since peptides with sample numbers #7, #9, #11, #12, #15, #16, #17, #18, #19, and #20 exhibited a rise in cytokine expressed upon stimulation via MHC class I, the peptides were determined as class I peptides.

The above immunoassay 2 is also a working example of information acquisition method of the present invention, in which a novel coronavirus is a target microorganism. Actually, the ratio of the quantitation value after infection to that before infection with a novel coronavirus was calculated in terms of each test peptide through the aforementioned step, whereby the MHC type of the test peptide was determined. As a result, each sample was successfully determined to be MHC class I or MHC class II.

1-14. (canceled)

15. An information acquisition method which comprises:
preparing [A] a trimer or hexamer of a composite protein represented by the following amino acid sequence of formula (1-2):
$${W}_{2} - {L}_{1} - {X}_{n} - Y \qquad ( 1 - 2 )$$
[wherein W₂ represents an amino acid sequence of a peptide or protein which originates from a pathogenic microorganism serving as a test immunogen; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):
$${Y}_{1} - {L}_{2} - {Y}_{2} - {Y}_{3} \qquad ( 2 )$$
[wherein Y₁ represents any one amino acid sequence selected from the group consisting of SEQ ID NOs: 2 to 5; Y₂ represents any one amino acid sequence selected from the group consisting of SEQ ID NOs: 6 to 9; L₂ represents a second linker sequence having 0 to 30 amino acids; Y₃ represents an amino acid sequence for modification; and either of Y₂ and Y₃ may be absent],
wherein the amino acid sequence represented by Xₙ, Y₁, or Y₂ may include a modified amino acid sequence thereof obtained by deleting, substituting, or adding one or more amino acid residues from, in, or to the original amino acid sequence;
immunizing a test animal with the trimer or hexamer [A]; removing immunocompetent cells of the immunized test animal from the body of the test animal; quantitating one or more physiologically active substances present in the separated immunocompetent cells; subsequently, infecting the separated immunocompetent cells with a target pathogenic microorganism; quantitating the physiologically active substances present in the infected immunocompetent cells; and acquiring information about secretion of the physiologically active substances after immunization with the test immunogen W₂, on the basis of, as an index, a change in each physiologically active substance level before and after infection obtained from the two quantitation values.

16. The information acquisition method according to claim 15, wherein the physiologically active substance includes a cytokine or a chemokine.

17. The information acquisition method according to claim 15 or 16, wherein the information about secretion of the physiologically active substances is information about whether the test immunogen W₂ is compatible with MHC class I, or MHC class II, or MH class I and MHC class II.

18. The information acquisition method according to claim 15, wherein the pathogenic microorganism is a virus.

19. A method for administering a component vaccine which comprises:
preparing a component vaccine containing, as an active ingredient, a protein trimer and/or a protein hexamer of a composite protein presented by the following amino acid sequence (1-1):
$$W - {L}_{1} - {X}_{n} - Y \qquad ( 1 - 1 )$$
[wherein W represents an amino acid sequence of a peptide including one or more peptides which are evaluated as an MHC class I compatible immunogen against a target pathogenic microorganism by an information acquisition method as recited in claim 15 and which originate from the pathogenic microorganism; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):
$${Y}_{1} - {L}_{2} - {Y}_{2} - {Y}_{3} \qquad ( 2 )$$
[wherein Y₁ represents any one amino acid sequence selected from the group consisting of SEQ ID NOs: 2 to 5; Y₂ represents any one amino acid sequence selected from the group consisting of SEQ ID NOs: 6 to 9; L₂ represents a second linker sequence having 0 to 30 amino acids; Y₃ represents an amino acid sequence for modification; and either of Y₂ and Y₃ may be absent],
wherein the amino acid sequence represented by Xₙ, Y₁, or Y₂ may include a modified amino acid sequence thereof obtained by deleting, substituting, or adding one or more amino acid residues from, in, or to the original amino acid sequence, and
administering to a subject the component vaccine to activate immune function by MHC class I compatible peptide against the target pathogenic microorganism in the subject.

20. A method for administering a component vaccine which comprises:
preparing a component vaccine containing, as an active ingredient, a protein trimer and/or a protein hexamer of a composite protein presented by the following amino acid sequence (1-1):
$$W - {L}_{1} - {X}_{n} - Y \qquad ( 1 - 1 )$$
[wherein W represents an amino acid sequence of a peptide including one or more peptides which are evaluated as an MHC class II compatible immunogen against a target pathogenic microorganism by an information acquisition method as recited in claim 15 and which originate from the pathogenic microorganism; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):
$${Y}_{1} - {L}_{2} - {Y}_{2} - {Y}_{3} \qquad ( 2 )$$
[wherein Y₁ represents any one amino acid sequence selected from the group consisting of SEQ ID NOs: 2 to 5; Y₂ represents any one amino acid sequence selected from the group consisting of SEQ ID NOs: 6 to 9; L₂ represents a second linker sequence having 0 to 30 amino acids; Y₃ represents an amino acid sequence for modification; and either of Y₂ and Y₃ may be absent],
wherein the amino acid sequence represented by Xₙ, Y₁, or Y₂ may include a modified amino acid sequence thereof obtained by deleting, substituting, or adding one or more amino acid residues from, in, or to the original amino acid sequence, and
administering to a subject the component vaccine to activate immune function by MHC class II compatible peptide against the target pathogenic microorganism in the subject.

21. A method for administering a component vaccine which comprises:
preparing a component vaccine containing, as an active ingredient, a protein trimer and/or a protein hexamer of a composite protein presented by the following amino acid sequence (1-1):
$$W - {L}_{1} - {X}_{n} - Y \qquad ( 1 - 1 )$$
[wherein W represents an amino acid sequence of a peptide including one or more peptides which are evaluated as an MHC class I and MHC class II compatible immunogen against a target pathogenic microorganism by an information acquisition method as recited in claim 15 and which originate from the pathogenic microorganism; L₁ represents a first linker sequence having 0 to 100 amino acids; X represents an amino acid sequence represented by SEQ ID NO: 1; Y represents an amino acid sequence of a cell introduction domain; and repetition number n of X is an integer of 1 to 10],
wherein the amino acid sequence of the cell introduction domain Y is represented by the following formula (2):
$${Y}_{1} - {L}_{2} - {Y}_{2} - {Y}_{3} \qquad ( 2 )$$
[wherein Y₁ represents any one amino acid sequence selected from the group consisting of SEQ ID NOs: 2 to 5; Y₂ represents any one amino acid sequence selected from the group consisting of SEQ ID NOs: 6 to 9; L₂ represents a second linker sequence having 0 to 30 amino acids; Y₃ represents an amino acid sequence for modification; and either of Y₂ and Y₃ may be absent],
wherein the amino acid sequence represented by Xₙ, Y₁, or Y₂ may include a modified amino acid sequence thereof obtained by deleting, substituting, or adding one or more amino acid residues from, in, or to the original amino acid sequence, and
administering to a subject the component vaccine to activate immune function by MHC class I and MHC class II compatible peptide against the target pathogenic microorganism in the subject.

22. The method according to claim 19, wherein, in the modified amino acid sequence obtained through deletion, substitution, or addition of one or more amino acid residues from, in, or to the amino acid sequence represented by Xₙ, Y₁, or Y₂ included in the above formulas, the number of modifications of amino acid residues in each amino acid sequence is ≤8n in the case of Xₙ; ≤30 in the case of Y₁; and ≤15 in the case of Y₂.

23. The method according to claim 19, wherein W serving as an immunogen is a peptide including two or more peptides, the two or more peptides being linked by the mediation of a linker.

24. The method according to claim 19, wherein the pathogenic microorganism is a virus.

25. The method according to claim 19, wherein the component vaccine is administered subcutaneously, intradermally, percutaneously, mucosally, or intramuscularly.

26. The method according to claim 19, wherein the component vaccine is administered to the nasal mucous membrane, throat mucous membrane, sublingual mucous membrane, or bronchial mucous membrane.

27. The method according to claim 26, wherein the component vaccine has a dosage form of spray, aerosol, or capsule.
